(19) United States
(12) Patent Application Publication
(10) Pub. No.: US 2011/0070219 A1
(43) Pub. Date: Mar. 24, 2011

(54) HIGH PRESSURE PROTEIN CRYSTALLIZATION

(76) Inventors: Matthew B. SEEFELDT, Boulder, CO (US); Theodore W. Randolph, (US); Ryan Crisman, (US)

(21) Appl. No.: 12/768,333

(22) Filed: Apr. 27, 2010

Related U.S. Application Data
(60) Provisional application No. 61/214,641, filed on Apr. 27, 2009.

(51) Int. Cl. A61K 38/47; C07K 1/14; C12N 9/24; A61K 38/27

(52) U.S. Cl. 424/94.61; 530/399; 435/200; 514/11.4

(57) ABSTRACT

The present disclosure provides an effective method for the crystallization of proteins at high pressure. A preferential excluding agent, and optionally other reagents may be incorporated into the method. The method is applicable to substantially all proteins.

### PRIORITY

This application claims priority to U.S. Provisional Application No. 61/214,641, filed Apr. 27, 2009, which is hereby incorporated by reference in its entirety.

### FIELD OF THE INVENTION

The field of the present invention is protein formulations and methods for making crystals of active molecules by subjecting the molecules to hydrostatic pressure in the presence of a preferentially excluding agent.

### BACKGROUND OF THE INVENTION

The formulation of therapeutic proteins and other macromolecules is an important field of research. Preferred attributes of an effective therapeutic formulation include stability and enhanced bioavailability. From a practical viewpoint, the methods for achieving these attributes must also be scalable and yield a consistent and safe protein product. One approach that allows for an extended release form of a protein-based drug involves the in vivo delivery of the protein in a crystalline state rather than in solution. Additional advantages of a crystalline preparation include the ability to administer a highly concentrated protein product and protection from chemical or physical degradation. It is also anticipated that protein crystal preparations (either dry or in a slurry formation) will require minimal excipients or carriers which often lead to adverse side effects.

The idea of a crystal-based peptide formulation has been present for some time with insulin crystals first being reported back in 1920s. Unfortunately, use of this technique for larger molecules (e.g. a protein with a molecular weight >10 K) has been unachievable at a manufacturing scale. Despite of the potential advantages, there is no commercially marketed formulation of a protein (here defined as a polypeptide with molecular weight greater than 10,000) where the protein exists physically in a crystalline state prior to administration. Thus, a need exists for a protein crystallization process that is suitable for scalable, e.g., commercial, manufacture in addition to providing a drug product that meets the regulatory requirements for human administration.

Advances in protein production and characterization have dramatically expanded the therapeutic applications of proteins. The success of these research and development efforts has brought about new and difficult challenges for the pharmaceutical industry. Protein therapeutics are complex molecules with marginal stability that are highly susceptible to the formation of non-native aggregates and precipitates (Cleland et al. (1993) Crit. Rev. Ther. Drug Carr. Syst. 10:307; Chang et al. (1996) Biophys. J. 71:3399). Hence, a major goal is to formulate a product such that it has a shelf-life of 12-24 months (Cleland et al. 1993) with minimal aggregate levels and high bioavailability.

Crystalline proteins may provide higher bioavailability, greater ease of handling, improved stability and altered dissolution characteristics (Hallas-Moller et al. (1952) Science 119:394; Hancock and Zografi (1997) J. Pharm. Sci. 86:1; Margolin and Navia (2001) Angew. Chem-Int. Edit. 40:2204). Physical and chemical degradation may be significantly reduced for proteins in their crystalline form, thereby protecting the therapeutic agent during processing, storage and after delivery (Basu et al. (2004) Protein Crystals for the Delivery of Biopharmaceuticals. Expert Opinion on Biological Therapy 4:301). In addition, protein crystals may allow for sustained and/or controlled release of the therapeutic agent for an effective duration, supporting malleable dosing regimens. Yet, despite the apparent advantages of crystalline formulations, insulin remains the only protein product produced and administered in crystalline form (Brange and Volund (1999) Advanced Drug Delivery Reviews 35:307).

The process of crystallization involves the steps of nucleation and crystal growth. The driving force for both nucleation and crystal growth is the degree of supersaturation, i.e., the concentration of the molecule in the solution above the equilibrium solubility value. If the degree of supersaturation is too low, spontaneous nucleation will not occur and crystallization will not take place. On the other hand, if the degree of supersaturation is too high, the molecules will form an amorphous precipitate. Thus, a supersaturation window exists in which the supersaturation is high enough to allow spontaneous nucleation and crystal growth to occur, yet low enough to avoid formation of amorphous precipitate.

A number of parameters can affect the degree of supersaturation and the process of crystal formation. These include, without limitation, the solubility and concentration of the molecule in the solution, the temperature, pH, viscosity, dielectric constant and ionic strength of the solution, as well as the presence of a precipitating agent, preferential excluding agent, reducing or oxidizing agent, nucleating agent, metal ion, detergent or amphiphile in the solution. Traditionally, precipitating agents such as sodium acetate or polyethylene glycol (PEG) have been used in a trial and error fashion to manipulate solubility and determine the conditions at which crystallization occurs. These agents compete with the molecule for water and exert excluded volume effects.

Pressure can also affect the solubility of a molecule and thus affect its crystallization. The first report on protein crystal growth under pressure (Vusuri et al., 1990) revealed that the yields of small glucose isomerase crystals could be enhanced with increasing pressure. In contrast, several high pressure crystallization studies with lysozyme reported that the solubility increased and the growth rate of the crystal and the nucleation rated decreased with increasing pressure. (Gross et al. (1991) FEBS Lett. 284:87; Suzuki et al. (1994) Jpn. J. Appl. Phys. 33:L1568-1570; Schall et al. (1994) J. Cryst. Growth 135:548-554; Saikumar et al. (1995) J. Cryst. Growth 151:173-179; Lorber et al. (1996) J. Cryst. Growth 158:103; Takano et al. (1997) J. Cryst. Growth 171:554-558; Suzuki et al. (2002) Bioch et Biophys Acta 1595:345-356). Similar observations of high pressure inhibition of crystal growth were also reported with subtilisin (Webb et al. (1999) J. Cryst. Growth 205:563-574; Waghmare et al. (2000) J. Cryst. Growth 208:676-686; Waghmare et al. (2000) J. Cryst. Growth 210:746-752)). The discrepancy between the results for glucose isomerase and other proteins suggests that pressure can have a variable and unpredictable effect on protein solubility, nucleation and crystal growth.

A crystalline formulation of a therapeutic protein (human growth hormone) has been reported previously (Govardhan et al. (2005) Pharm. Res. 22(9):1461). The protein crystals that formed at atmospheric conditions in the presence of PEG were found to have undesirable acicular or needle-like morphologies. In addition, the crystals required a polyelectrolyte coating to slow the dissolution rate in vivo. Therefore, there remains a need for a broadly applicable and scalable process that is capable of producing a crystal-based protein formulation with sufficient yields and high purity. High hydrostatic pressure has a profound impact on reaction kinetics, thermodynamics, and compound physical properties (Gross and Jaenicke (1994) Eur. J. Biochem. 221:617). Reactions with negative activation volumes (kinetics) and decreases in system volumes (thermodynamics) are favored at elevated pressures. In addition, pressure can alter phase behavior and structure of water (Giovambattista et al. (2006) Physical Review E. 73:4) and is a readily scalable process. However, in comparison to temperature, the development of pressure as a process parameter has been explored to a much lower extent. This is due to the difficulties in developing equipment which can withstand the large forces generated in pressure applications.

The inability to perform static light scattering measurements at elevated pressure has led to a lack of understanding of the pressure effects on hydration, protein-protein interactions and protein dissociation at elevated pressures. Data from static light scattering measurements performed at elevated pressures support the advantages of protein refolding at elevated pressures. The field of high-pressure bioscience has experienced an amazing growth in terms of techniques and applications toward understanding the pressure effects on proteins. Examples of spectroscopic techniques to follow protein structural changes, in-situ, as a function of pressure include fluorometry (Paladini and Weber 1981), UV/Vis (Webb et al. 2000; Lange and Balny 2002), NMR (Jonas and Jonas (1994) Annu. Rev. Biophys. Biomolec. Struct. 23:287) and FTIR (Goossens et al. (1996) Eur. J. Biochem 236:254). In addition, protein crystallization at elevated pressures (Visuri et al. (1990) Nature-Biotechnology 8:547; Webb et al. 1999; Waghmare et al. 2000; Suzuki et al. 2002) has provided insight in to pressure effects on protein solubility and has even shown a potential advantage in the crystal growth process (Visuri et al. 1990). Lastly, high hydrostatic pressures have been used to denature proteins, accelerate enzyme kinetics, dissociate native oligomers (see review by Gross and Jaenicke (Gross and Jaenicke 1994) in addition to being an effective alternative strategy for refolding a number of protein aggregates at high concentration and with high yield (St John et al. (1999) PNAS 96:13029; St John et al. (2001) J. Bio. Chem. 276:46856).

### SUMMARY OF THE INVENTION

Crystallization of recombinant proteins as a function of pressure with PEG as the precipitating agents finds that pressure inhibits crystal formation at lower PEG concentrations whereas increasing PEG concentration created solution conditions that favored the formation of crystals at elevated pressures. At atmospheric pressure, amorphous precipitate formed in the same solutions conditions. High pressure analytical techniques determined that the decrease in the excluded volume interactions with increasing pressure reduces the thermodynamic driving force for protein crystallization. Increasing the concentration of PEG and protein in solution resulted in an increase in thermodynamic instability resulting in solution conditions that favored crystal formation, over amorphous precipitate, at elevated pressures.

Kinetics of rhGH crystallization at elevated pressures was determined from particle sizing data. An increase in crystallization rate occurred at 250 MPa, relative to crystal formation at 0.1 MPa. Further investigation determined that the increase in crystallization rate is likely due to the increase in the growth rate constant at the higher pressure. Bulk diffusion, adsorption and surface diffusion were discussed as potential reasons for the increase in growth rate constant at elevated pressures. We speculate that the pressure effects on the non-covalent surface interactions (e.g. hydrophobic and electrostatic) increase the surface adsorption and diffusion, ultimately increasing the crystallization rate.

### BRIEF DESCRIPTION OF THE FIGURES

FIG. 1 Panel A. Crystal formation at ambient pressure after 14 hours. Panel B. The same solution conditions were held at 2 kbar for 14 hours. It appears that high pressure arrested crystallization at these solution conditions (15 mg/ml rhGH, pH 8.6, 100 mM Tris, 6% PEG-6000, 500 mM sodium acetate, 25 C).

FIG. 2 Panel A. Solubility of crystals as a function of pressure in the absence of PEG-6000 (pH 8.6, 100 mM Tris, 500 mM sodium acetate, 25° C.). Panel B. The natural log of solubility as a function of pressure. Table insert shows the supersaturation and volume change determined from the solubility of the protein and the slope of the line of the natural log of solubility versus pressure, respectively.

FIG. 3 shows the hexagonal crystals and amorphous precipitate of rhGH produced in accordance with this invention in the presence of 8% PEG-6000, at pH 7.0.

FIG. 4 shows the solubility of rhGH plotted as a function of pressure in the absence of PEG-6000, at pH 7. Table insert shows the supersaturation and volume change values at atmospheric pressure and at 2 kbar pressure.

FIG. 5 shows the apparent solubility of rhGH as a function of pressure in the absence of (closed diamonds) and presence of (open squares) 8% PEG-6000.

FIG. 6 shows the supersaturation values and crystallization results obtained for rhGH at atmospheric and elevated pressures.

FIG. 7 shows the concentration of rhGH in the supernatant during the formation of crystals obtained in accordance with the present invention.

FIG. 8 shows the natural log of rhGH apparent solubility as a function of pressure in 50 mM Tris, 0.5M NaAc, pH 7.5 at 25° C. in the absence of PEG-6000.

FIG. 9 shows static light scattering data for rhGH at 0.1 MPa (diamond) and 250 MPa (triangle) in 50 mM Tris, 0.5M NaAc, pH 7.5 at 25° C. in the absence (A) and presence (B) of 6% PEG-6000.

FIG. 10 shows (A) static light scattering data of PEG-6000 at varying pressures, and (B) the apparent hard sphere radius (R₃) of PEG-6000 as a function of pressure.

FIG. 11 shows rhGH activity as a function of PEG-6000 concentration at 0.1 MPa (⋄) and 250 MPa (▴) in 50 mM Tris, 0.5M NaAc, pH 7.0, 25° C. at rhGH concentrations of 15 mg/mL.

FIG. 12 shows batch crystallization attempts for recombinant human growth hormone. (A) Successful crystal formation at 0.1 MPa and (B) no crystal formation at 250 MPa. Solution conditions containing 15 mg/mL rhGH, pH 8.6, 100 mM Tris, 500 mM sodium acetate, at 25° C. in the presence of 6% PEG-6000. (C) Successful crystal formation at 250 MPa and (D) amorphous precipitate formation at 0.1 MPa and in solution conditions containing 35 mg/mL rhGH, 50 mM Tris, 500 mM sodium acetate pH 7.0 at 25° C. in the presence of 8% PEG-6000.

FIG. 13 shows the crystals of xylanase produced in accordance with the present invention.

FIG. 14 shows the supersaturation values and crystallization results obtained for xylanase at atmospheric pressure (diagonal stripes) and at a pressure of 1 kbar (vertical stripes).

FIG. 15 shows the solubility of xylanase plotted as a function of pressure in the presence of varying amounts of PEG-6000. The solubility of xylanase (13.5 mg/ml) as a function of pressure in the presence of varying amounts of PEG-6000 (diamond 10%, square 20%, triangle 22.5%,-25%).

FIG. 16 shows the solubility of xylanase plotted as a function of pressure in the absence of PEG-6000 at varying concentrations of xylanase.

FIG. 17 shows the effect of pressure on a solution containing a mixture of amorphous precipitate and crystals of xylanase.

FIG. 18 shows the concentration of xylanase obtained in the supernatant when a solution containing a mixture of amorphous precipitate and crystals formed at atmospheric pressure was subjected to varying pressures of 1-3 kbar for 24 hours.

FIG. 19 shows experimental concentration profile at respective crystallization conditions.

FIG. 20 shows total number of crystals per mL of solution at respective crystallization conditions.

FIG. 21 shows particle size of rhGH crystals in respective solution conditions.

### DETAILED DESCRIPTION

Reference will now be made in detail to representative embodiments of the invention. While the invention will be described in conjunction with the enumerated embodiments, it will be understood that they are not intended to limit the invention to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

All publications and patents mentioned herein are incorporated herein by reference in their respective entireties for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventor is not entitled to antedate such disclosure by virtue of prior invention.

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices and materials are now described.

The present invention describes methods for the crystallization of a macromolecule that involve the addition of a preferential excluding agent such as polyethylene glycol to the solution and subjecting the solution to high hydrostatic pressure.

Pressure typically increases the solubility of a molecule in solution and thus, adversely affects the kinetics of nucleation and/or growth of crystals. However, it has been found that the combination of a preferential excluding agent and high pressure results in the formation of high quality crystals. At atmospheric pressure the same solution conditions produced an amorphous precipitate (Hancock and Zografi (1997) J. Pharm. Sci. 86(1):1-12). Therefore, the application of pressure is capable of modulating the solubility of the protein in solution, in addition to affecting the overall solution interactions such that crystal formation is favored over precipitation.

The present invention provides methods for the production of large, well dispersed and homogeneous crystals that are essentially devoid of amorphous precipitate. As disclosed, it is also possible to use pressure to enhance the purity of a heterogeneous protein preparation. Pressurization of a mixture of amorphous precipitate and crystals results in the disaggregation/solubilization of the precipitate while the protein crystals remain in solution. Presumably, the irregular arrangement of molecules in a less dense amorphous state has a larger specific volume than if the molecules were arranged in a crystal lattice formation. The amorphous precipitate is thus more susceptible to the effects of pressure while the crystals remain intact (Hancock et al., 1997). The ability to preferentially dissociate amorphous precipitate while maintaining, and possibly forming crystals at elevated pressures has not been previously reported.

The driving force for protein nucleation and crystallization is the degree of supersaturation (Ducruix and Giege (2000) Crystallization of Nucleic Acids and Proteins: A Practical Approach, 2ed. Oxford University Press, USA, pp. 464). A saturated solution contains an amount of solute such that neither growth nor dissolution will occur upon the addition of crystals to the solution. This corresponds to a thermodynamic equilibrium between the two phases (crystalline and solution) such that the chemical potential of each species ‘i’ is the same in both phases (μ_{ic}=μᵢₛ), where μ_{ic} is the chemical potential of species i in the crystal and μᵢₛ is the chemical potential of the solution, such that:

μ_{ic}=μᵢₛ=μᵢ₀+RTlnaᵢ=μᵢ₀+RTln(γcᵢ) (1)

Where μᵢ₀ is the standard chemical potential, aᵢ is the activity, γ the activity coefficient, and cᵢ the concentration of the species i.

Supersaturation is reached when the chemical potential of the solute in solution is greater than that in the crystal. Supersaturated conditions may be achieved by varying parameters that affect the chemical potential (e.g. temperature, protein concentration, salt concentration, addition of cosolutes, pressure) (Ducruix and Giege 2000). In general, high degrees of supersaturation (between 2-10× solubility) are required to initiate nucleation of protein crystals. (Ducruix and Giege 2000). On the other hand, if the degree of supersaturation is too high, the supersaturated protein molecules separate so rapidly from the supersaturated solution that an amorphous precipitate forms. Thus, a “supersaturation window” exists in which the protein concentration is high enough to allow spontaneous nucleation and crystal growth to occur, yet low enough to avoid formation of amorphous precipitate.

One of the most common additives used to foster protein crystallization is polyethylene glycol (PEG) (McPherson (1990) Eur. J. Biochem. 189:1). PEG is a hydrophilic nonionic polymer used in many biochemical and pharmaceutical applications. In protein crystallography, it is generally believed that the main mechanism of action of PEG on proteins can be described through the influence of mutual volume exclusion on the entropy of the system (Bhat and Timasheff (1992) Protein Sci. 1:1133; Adams and Fraden (1998) Biophys. J. 74:669; Tardieu et al. (2002) Acta Crystallogr. Sec. D: Biol. Crystallogr. 58:1549; Wang and Annunziata (2007) J. Phys. Chem. 111:1222). This mechanism is usually denoted using the terms depletion interactions or macromolecular crowding (Wang and Annunziata 2007). This exclusion gives rise to an effective interaction between the protein particles (Asakura and Oosawa (1954) J. Chem. Phys. 22:1255) and has been determined from x-ray (Vivares et al. (2002) European J. Physics 9) and light scattering (Asakura and Oosawa 1954; Bloustine et al. (2006) Physical Review Letters 96) measurements.

Pressure is known to affect the chemical potential of a protein system and, hence, protein crystallization. The first report on protein crystal growth under pressure revealed that the yields of small glucose isomerase crystals could be enhanced with increasing pressure (Visuri et al. 1990). Glucose isomerase is the only protein to date for which it has been reported that high pressures enhance crystallization. In contrast, several studies of lysozyme crystallization at high pressures reported that lysozyme solubility increases and crystal nucleation and growth rates decrease with increasing pressure (Suzuki et al., 1994; Schall et al., 1994; Saikumar et al., 1995; Lorber et al., 1996; Takano et al., 1997; Suzuki et al. (2002) Crystal Grown and Design 2:321). Similar observations of high pressure inhibition of crystal growth were also reported with subtilisin (Webb et al., 1999; Waghmare et al., 2000a; Waghmare et al., 2000b).

The effects of pressure on the crystallization of recombinant protein, e.g., human growth hormone (rhGH), in the presence of a preferential excluding agent, e.g., PEG, are disclosed herein. rhGH is used as an illustrative example because of both the known set of conditions that result in crystallization at atmospheric pressure and also the numerous potential therapeutic advantages of an extended release formulation for rhGH (Govardhan et al. 2005). The increase in supersaturation in the presence of PEG results in an increase in protein thermodynamic non-ideality based on excluded volume contributions. It appears that pressure decreases the PEG-induced thermodynamic non-ideality of the solution, relative to atmospheric pressure, reducing the thermodynamic driving force for protein crystallization. Therefore, increasing the concentration of PEG and/or protein in solution results in an increase in thermodynamic instability resulting in solution conditions that favor crystal formation at elevated pressures. The ability to control protein supersaturation levels by adjusting the pressure has broad potential applicability to recombinant proteins in that it both optimizes current protein crystallization processes and provides novel crystallization conditions.

In one embodiment, the present invention includes a method for forming crystals of a macromolecule in a solution. This method includes adding a volume excluding excipient to the solution and applying to the solution a hydrostatic pressure to enhance the formation of crystals. The method also includes depressurization of the solution.

The method described herein involves raising the pressure above atmospheric pressure. Atmospheric pressure is approximately 15 pounds per square inch (psi) or 1 bar. In methods of the current invention, pressure may be generated using techniques and equipment known in the art for creating hydrostatic pressure. For example, hydraulic intensifier equipment may be used to create hydrostatic pressure on proteins. The ability to vary the rate of pressurization (from 10 minutes to 48 hour) also allows one to control the thermodynamic parameters of the solution which is useful when studying crystal growth or proteins in the partially unfolded states.

Hydrostatic pressure has been shown to be an effective refolding tool, enabling protein renaturation at relatively high concentrations and with high yields. Such methods of refolding proteins using elevated hydrostatic pressure on solutions of proteins in order to disaggregate, unfold, and properly refold proteins are described in U.S. Pat. No. 6,489,450, U.S. Pat. No. 7,064,192, U.S. Patent Application Publication No. 2004/0038333, and International Patent Application WO 02/062827, each of which is incorporated by reference herein in their entirety. Certain devices have been developed which are particularly suitable for refolding of proteins under elevated pressure as well as performing solution exchange under pressure; see International Patent Application Publication No. WO 07/062,174, which is incorporated by reference herein in its entirety. Hydrostatic pressure has also been shown to enable derivatization of proteins to form biologically active polymer-protein or cytotoxic agent-protein conjugates. Such methods have been described in Provisional application 61/057,731, which is incorporated by reference herein in its entirety.

Pressure vessels ranging from 150 mL to 10 liters are commercially available (BaroFold, Inc). Several vendors (cf. NCI Hyperbaric, Avure, and High Pressure Equipment Co.) produce large volume (>600 L) high pressure systems currently commercially used in the food industry for, e.g., pasteurization. Such equipment may be adapted for use with the disclosed methodologies, thus, enabling high pressure treatment to be readily applicable for the large scale manufacture of crystal biopharmaceutical formulations.

In some embodiments, the crystal inducing excipient may be sodium chloride, sodium acetate, sodium phosphate, potassium phosphate, sodium citrate ammonium sulfate, ethanol, glycine-HCl, hydroxyl ethyl starch, heptane 1,2,3-triol, polyethylene glycol (PEG), or dextran.

In a preferred embodiment, the crystal inducing excipient is a preferential excluder, e.g., PEG (Bhat et al., 1992). The term PEG or polyethylene glycol refers to a polymer of ethylene oxide molecules and includes polymer molecules of varying polymer lengths and molecular weights. For example, PEG molecules are currently commercially available over a wide range of molecular weights ranging from about 100 to about 50,000,000 Daltons. Furthermore, PEG molecules may have different geometries, and for example, may be linear or branched. The term PEG may also refer to modified forms of PEG that are obtained, depending on the initiator used in the polymerization process, such as methoxy polyethylene glycol or mPEG. The term PEG or PEG molecules as used herein encompasses all forms of PEG molecules known in the art. In preferred embodiments, PEG has a molecular weight ranging from about 50 Daltons to about 500,000 Daltons, from about 75 Daltons to about 100,000 Daltons, from about 100 Daltons to about 30,000 Daltons, from about 1,000 Daltons to about 20,000 Daltons, or from about 3,000 Daltons to about 12,000 Daltons. For the present invention, a target PEG range may include 100-20K MW, and a preferred PEG may include 3350-10K MW.

One of the method embodiments of the present invention further comprises depressurizing the solution. The step of depressurizing may be conducted at a suitable rate to control the size, morphology and/or degree of homogeneity of the crystal. Depressurization rates may vary over a wide variety of ranges from about 25,000 bar per minute to about 1 bar per minute. In various embodiments the lower end of the range may be selected from about 1, 25, 50, 100, 200, 300, 400, 500, 600, 700, 800, 900, 1000, 2000, 3000, 4000 and 5000 bars per minute and the upper end of the range may be selected from about 25000, 15000, 10000, 5000, 4000, 3000, 2000, 1000, 900, 800, 700, 600, 500, 400, 300, 200 and 100 bars per minute. These embodiments may include any one of the lower limits and any one of the upper limits. In some embodiments, the depressurization rate may range from about 1 bar per minute to about 20 bar per minute, from about 5 bar per minute to about 15 bar per minute, or from about 8 bar per minute to about 12 bar per minute. In some embodiments, the rate of depressurization may range from about 100 to about 300 bars per minute over a 10 minute depressurization period.

In some embodiments, when the solution is at atmospheric pressure crystals are not formed. In some embodiments, at atmospheric pressure an amorphous precipitate may be formed, while in some embodiments the molecule may remain dissolved in the solution. In some embodiments, crystals are not formed even when the supersaturation value of the solution at atmospheric pressure is the same as that at the elevated pressure. The supersaturation value may be held constant, for instance, by changing the concentration of the molecule in the solution.

With the methods described herein it is possible to obtain highly pure crystals. In various embodiments, the amount of amorphous precipitate formed when pressure is applied to the solution may be less than 25%, 20%, 15%, 10%, 9%, 8%, 7%, 6%, 5%, 4%, 3%, 2% or 1% of the amount of crystals. Depending on the protein sample, the amount of precipitate formed can vary depending on the amount of pressure that is applied to the solution. For example, in studies with xylanase, protein samples contained less than 15% precipitate when subjected to 1000 bar and less than 5% when subjected to 1500 bar of pressure. In contrast, rhGH pressure studies revealed that the amount of amorphous precipitate formed is equal to or less than about 2%, regardless of the pressure.

The crystals formed using the methods of the instant invention may differ qualitatively from the crystals formed when the solution is at atmospheric pressure. For instance, the crystals may have a different morphology, may be greater in number or larger in size, or may be more homogeneous.

The method may further comprise the step of recovering the crystals after the step of depressurizing. The crystals may be recovered by methods conventionally used for recovery of the crystals. The crystals may be stored as a slurry, or may be lyophilized and stored in dry form.

The methods of the present invention may be used to crystallize any macromolecule. The molecule may be a protein, DNA, RNA, carbohydrate, peptide or polymer. As used herein, the term protein is defined as a polypeptide having a molecular weight greater than about 10,000. Classes of proteins include, without limitation, globular, fibrous, and membrane proteins.

Typically, the methods of the invention described herein are applied to solutions or mixtures where the total protein concentration is in the range from about 0.001 mg/ml to about 1000 mg/ml, from about 0.1 mg/ml to about 500 mg/ml or from about 1 mg/ml to about 100 mg/ml. [Note: illustrative examples include rhGH 35 mg/ml, xylanase 13.5 mg/ml]. The solution may comprise additional components such as a buffer or a salt. The buffer may be acetate, citrate, phosphate, sulfate, Tris or any small molecule that is capable of controlling the pH of the solution. The salt may be, e.g., magnesium chloride, sodium chloride or sodium acetate.

The methods of the present invention may be used to crystallize any protein, including antibodies, antibody fragments, trophic factors, cytokines, lymphokines, toxoids, growth factors, hormones, human growth hormones, growth hormone family members, nerve growth hormones, fertility hormones, postridical hormones, fusion proteins, glycoproteins, synthetic antigens, recombinant antigens, histocompatibility antigens, viral surface proteins, bone morphogenic proteins, enzymes, blood clotting factors, adhesion molecules, multidrug resistance proteins, interleukins, interleukin receptors, chemokines, interferon receptors, T-cell receptors, blood factors, leukocyte markers, monocyte-macrophage colony stimulating factors, granulocyte colony stimulating factors, integrins, selectins, and lectins. Specific examples include but are not limited to erythropoietin, Factor VIII, insulin, amylin, TPA, dornase-α, α-1-antitrypsin, urease, FSH, LSH, tetanus toxoid, diptheria toxoid, glucagon-like peptide 1, TGF-β, α-IFN, γ-IFN, TNF, lymphotoxin, Migration inhibition factor, neuregulin, CD2, CD3, CD4, CD5, CD6, CD7, CD8, CD11a, CD11b, CD11c, CD13, CD14, CD18, CD19, CE20, CD22, CD23, CD27, CD28, B7.1, B7.2, B7.3, CD29, CD30, CD40, gp39, CD44, CD45, Cdw52, CD56, CD58, CD69, CD72, CTLA-4, LFA-1, SLex, SLey, SLea, SLeb, VLA-1, VLA-2, VLA-3, VLA-4, VLA-5, VLA-6, LFA-1, Mac-1, p150, p95, L-selectin, P-selectin, E-selectin, VCAM-1, ICAM-1, ICAM-2, LFA-3, IL-1, IL-2, IL-3, IL-4, IL-5, IL-6, IL-7, IL-8, IL-9, IL-10, IL-11, IL-12, IL-13, IL-14, IL-15, IL-1R, IL-2R, IL-4R, IL-5R, IL-6R, IL-7R, IL-8R, IL-10R, IL-11R, IL-12R, IL-13R, IL-14R, IL-15R, PF4, MIP1a, MCP1, NAP-2, Groα, Groβ, IL-8, TNF α, TGF β, TSH, VEGF/VPF, PTHrP, EGF, PDGF, endothelin, gastrin releasing peptide (GRP), TNFαR, RGFβ R, TSHR, VEGFR/VPFR, FGFR, EGFR, PTHrPR, PDGFR, EPO-R, GCSF-R, IFNα R, IFNβR, IFNγR, IgE, FceRI, FceRII, complement C3b, complement C5a, complement C5b-9, Rh factor, fibrinogen, fibrin, myelin associated growth inhibitor, prolactin, placental lactogen, thrombopoietin, oncostatin M, ciliary neurotrophic factor (CNTF), leukemia inhibitory factor (LIF), epsilon interferon, omega interferon, tau interferon, granulocyte-colony stimulating factor (G-CSF), granulocyte-macrophage colony stimulating factor (GM-CSF), macrophage colony stimulating factor (M-CSF), cardiotrophin-1 (CT-1), recombinant human Growth Hormone (rhGH) or xylanase. Suitable proteins also include any of the foregoing proteins or classes of proteins that have been modified, such as by deletions, substitutions or additions of amino acids, including without limitation, the introduction of functional groups.

In preferred embodiments, the protein may be rhGH or xylanase. In one embodiment, the protein is rhGH and the crystals have hexagonal morphology.

In some embodiments, the crystals retain the biological activity of the molecule. Biological activity of a molecule as used herein, means at least about 10%, at least about 20%, at least about 30%, at least about 40%, at least about 50%, at least about 60%, at least about 70%, at least about 80%, at least about 90%, or at least about 95% of maximal known specific activity as measured in an assay that is generally accepted in the art to be correlated with the known or intended utility of the molecule. For molecules intended for therapeutic use, the assay of choice may be one accepted by a regulatory agency to which data on safety and efficacy of the molecule is submitted. A molecule having greater than 10% of maximal known specific activity is “biologically active” for the purposes of the invention.

In some embodiments, the instant method further comprises a step selected from the group consisting of: changing the pH of the solution, changing the temperature of the solution, changing the dielectric constant of the solution, changing the viscosity of the solution, changing the ionic strength of the solution, changing the concentration of the molecule, adding a reducing agent to the solution, adding an oxidizing agent to the solution, adding a nucleant to the solution, adding a metal ion to the solution, adding a detergent to the solution and adding an amphiphile to the solution. An amphiphile is a molecule that possesses both hydrophilic and hydrophobic properties.

Another embodiment of the present invention includes a method of forming crystals of a molecule, which includes applying to the solution a hydrostatic pressure of about 0.1 to about 25 kbars. The method also includes adding a crystal inducing agent or preferential excluding agent to the solution. The method also includes depressurizing the solution. In some embodiments, the pressure is sufficient to form crystals of the molecule. Preferred embodiments may include applying pressures of about 0.5 to about 10 kilobars, of about 0.75 to about 5 kilobars, or of about 1 to about 3 kilobars.

[Examples: rhGH 2 kbar, xylanase 1 or 1.5 kbar]

Another embodiment of the present invention includes purifying a composition comprising crystals of a molecule and amorphous precipitate of the molecule. The method comprises the steps of applying hydrostatic pressure to the composition comprising the crystals and the amorphous precipitate to dissolve at least a portion of the amorphous precipitate while maintaining at least a portion of the crystals and depressurizing the composition. In some embodiments, more than about 10%, 20%, 30%, 40%, 50%, 60%, 70%, 80%, 90%, 91%, 92%, 93%, 94%, 95%, 96%, 97%, 98% and 99% of the amorphous precipitate is dissolved.

During depressurization the solution is brought to atmospheric pressure. Depressurization rates may vary over a wide variety of ranges from about 25,000 bar per minute to about 1 bar per minute. In some embodiments, the rate may vary from about 100 to about 300 bars per minute.

In some embodiments, the amount of crystals formed after applying the pressure may be greater than before applying the pressure. Presumably, pressure treatment causes the disaggregation/solubilization of the amorphous precipitate. Once in solution, the molecules are now able to enter the energetically favorable crystal state.

In some embodiments, the method may further include a step such as adding a precipitating agent to the solution, changing the pH of the solution, changing the temperature of the solution, changing the dielectric constant of the solution, changing the viscosity of the solution, changing the ionic strength of the solution, changing the concentration of the molecule, adding a reducing agent to the solution, adding an oxidizing agent to the solution, adding a nucleant to the solution, adding a metal ion to the solution, adding a detergent to the solution, or adding an amphiphile to the solution. In preferred embodiments, the method includes adding the preferential excluding agent, (PEG). In some embodiments, the crystals may be biologically active.

Another embodiment of the present invention includes a method for purifying a composition comprising crystals and amorphous precipitate of a molecule comprising applying to the solution a hydrostatic pressure of about 100 to about 25000 bars, 250 to about 10000 bars, and 500 to about 5000 bars. Preferably in the range of 750-3000 bar.[range 1000-3000 bars] The method further includes depressurizing the composition.

Other embodiments of the present invention include compositions comprising crystals made by the methods described herein. Still other embodiments of the invention include pharmaceutical compositions comprising crystals made by the methods described herein and a pharmaceutically acceptable excipient. Such excipients may include, without limitation, diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, chelating agents and the like; used either alone or in combination thereof. In other embodiments the invention includes methods of treatment of a subject comprising the step of administering to the subject a therapeutic formulation comprising the crystals made by the methods described herein.

High pressure protein crystallization has been studied as a batch process. Atmospheric batch crystallization attempts have yielded numerous successful crystallization attempts. However, high pressure batch studies have not been as reluctant. One of the biggest hindrances to high pressure crystallization is that pressure has shown to increase the solubility of most proteins, ultimately decreasing the driving force for crystallization (Webb et al. 1999; Suzuki et al. 2002a). Therefore, the ability to adjust solution conditions while at elevated pressure could provide a new process for producing novel, high quality protein crystals.

The ability to perform solution exchange at elevated pressures would provide many advantages to overcome the drawbacks to the current batch high pressure processing of proteins. For starters, when attempting to refold a protein aggregate or inclusion body it would be advantageous to augment the dissolution step of pressure-modulated refolding with cosolutes that promote aggregate dissociation. For example, using a two-part process wherein protein aggregates are first dissociated under pressure in solution conditions that enhance aggregate dissolution (e.g., arginine, urea, GdnHCl), followed by a second step where the solution conditions are changed, while still under pressure, to conditions that foster protein folding and native assembly (e.g., 0.5 M sucrose).

Another example of the benefits of solution exchange while at elevated pressure is the ability to adjust protein solubility and, hence, supersaturation levels which is the driving force for protein crystallization. The supersaturation level has to be high enough such that spontaneous nucleation occurs yet low enough such that formation of amorphous precipitate does not form. Once nucleation occurs, crystal growth continues until equilibrium between protein in solution and crystal is reached. One way to decrease the solubility of the protein would be to adjust the pH toward the isoelectric point. One potential advantageous process, using high pressure solution exchange, would be to start the crystal nucleation and growth at elevated pressure in solution conditions that favor spontaneous nucleation. Once crystal growth begins, a change in solution conditions (e.g. change in pressure or pH) to decrease solubility of the protein in solution could drive the equilibrium toward the crystal form and ultimately result in higher yields of the target crystal. The model of solution exchange at high pressure is disclosed in WO 07/______.

A high pressure chamber able to be subjected to high pressures (up to 3000 bar) containing two primary containers whose contents are emptied and mixed into a secondary container through the use of fluid hydraulics was developed and utilized to illustrate non-batch process crystallization. This device enables solution exchange, and thus, the adjustment of solution conditions during pressure treatment.

The following examples are provided for illustrative purposes, and are not intended to limit the scope of the invention as claimed herein. Any variations which occur to the skilled artisan are intended to fall within the scope of the present invention. All references cited in the present application are incorporated by reference herein to the extent that there is no inconsistency with the present disclosure. The invention will now be further described with respect to the following illustrative examples.

### Example 1

### Production of the rhGH Crystals at High Pressure

Crystallization of recombinant human growth hormone (rhGH) at elevated pressure was accomplished in the present of 8% PEG, whereas amorphous precipitate formed in the same solution conditions at atmospheric pressure.

PEG-6000, sodium acetate (NaAc) and tris(hydroxymethyl) aminomethane (Tris) were purchased from Sigma-Aldrich (Sigma-Aldrich, St. Louis, Mo.). The protein recombinant human growth hormone (rhGH, Saizen®) was purchased as a lyophilized powder (Serono Inc., Geneva, Switzerland). rhGH was reconstituted in bacteriostat water to a final concentration of 8.8 mg/mL. The reconstituted protein was dialyzed into appropriate buffer and concentrated using 5 kDa MWCO Amicon Ultra-4 centrifugal filter device (Millipore Corp., Bedford, Mass.). All solutions were filtered with a 0.22 μm Millex GV filter unit (Millipore Corp., Bedford, Mass.) with the exception of PEG-6000. Each sample was loaded into 1 mL BD plastic syringe with a heat sealed tip and the plunger was re-inserted. The configuration of this device allows the sample to handle high pressures. Pressure was generated using a custom-built assembly consisting of an electric powered motor which turns a lead-screw driven piston pump with water as the pressure transmitting fluid. A custom-built pressure cell was attached to the pressure generator.

Batch crystallization studies of rhGH were performed in 1 mL BD syringes at 25° C. over 16 hours. The samples were visually analyzed using a Stereomaster Digital Zoom Microscope (Fisher Scientific, Pittsburgh, Pa.). Solubility data were collected by centrifuging the batch sample and measuring the protein concentration of the supernatant using UV-spectrophotometry at 280 nm. Crystallization screens were performed at 1 bar and 2000 bar over a broad range of solution conditions (10-40 mg/mL rhGH, pH 7.0-8.6, 0-0.5 M NaAc, 0-20% PEG-6000 in 100 mM Tris-HCl).

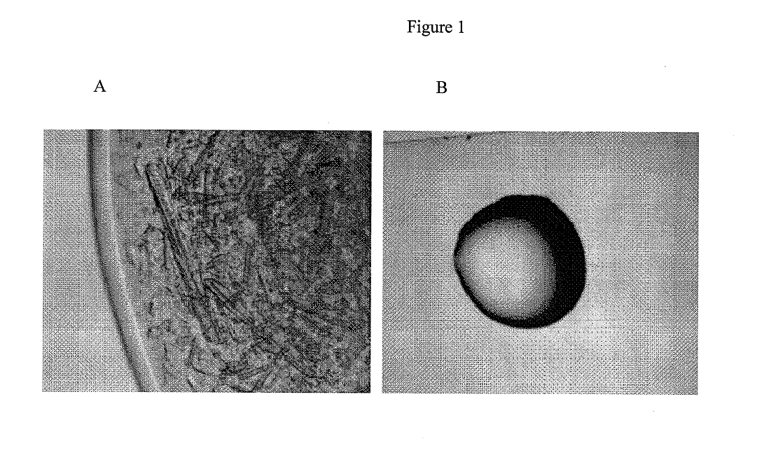

Under the solution conditions of 15 mg/mL rhGH, pH 8.6, 100 mM Tris, 500 mM sodium acetate, at 25° C. at atmospheric pressure (1 bar) in the presence of 6% PEG-6000 rod-like crystals of rhGH formed. Under similar solution conditions at the pressure of 2 kbar, no crystal formation was observed. (See FIG. 1)

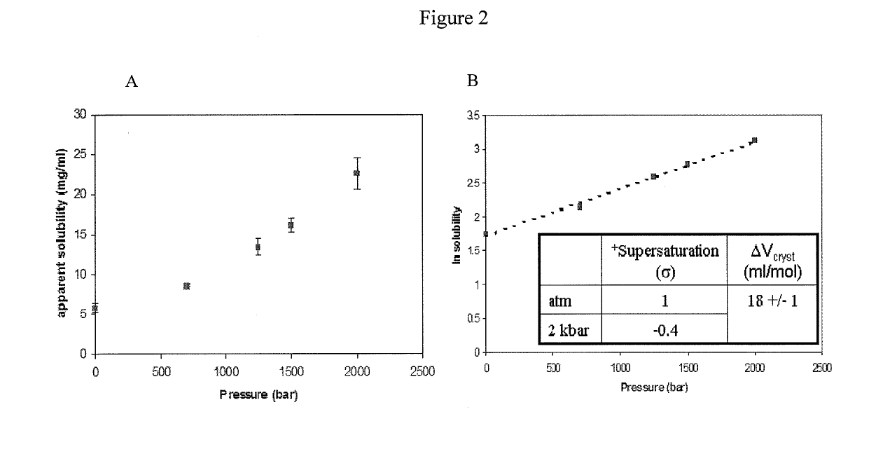

The apparent solubility of rhGH increases as a function of pressure which presumably explains the arrest in crystallization at elevated pressure. FIG. 2 A shows the solubility of rhGH plotted as a function of pressure in the absence of 6% PEG-6000. [The disclosure says “in the absence of PEG,” but then lists 6% PEG in the solution. FIG. 2B shows the graph of natural log of solubility plotted as a function of pressure. The inset table shows the supersaturation and volume change values at atmospheric pressure and at 2 kbar pressure. At 2 kbar the protein in solution is no longer supersaturated. At constant temperature, the volume change of crystallization with pressure is related to the change in the equilibrium constant K between protein in solution and protein in the crystalline state. This relation is given by

$${[ \frac{\partial ln K}{\partial P} ]}_{T , n} = - \frac{Δ V}{RT} \qquad ( 1 )$$

where ΔV is the volume change of crystallization (cm³ mol⁻¹), R the gas constant (cm³ MPa mol⁻¹ K⁻¹), T the absolute temperature (K) and P the pressure (MPa). The positive volume change associated with protein crystallization is associated with the increase in protein solubility as a function of pressure, resulting in the arrest in crystallization occurring at 2 kbar. (Please explain.)

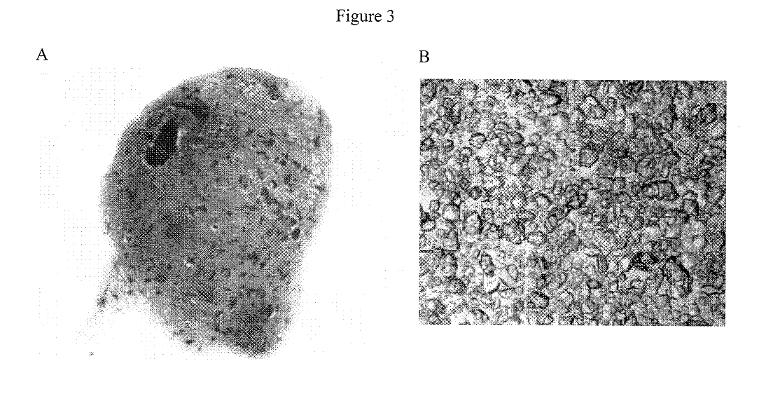

A variety of solution conditions at 1 bar and 2 kbar were evaluated to identify a pressure dependent “crystallization window”. At a protein concentration of 35 mg/mL rhGH, in pH 7.0, 100 mM Tris, 500 mM sodium acetate at 25° C. in the presence of 8% PEG-6000, formation of amorphous precipitate occurred at atmospheric pressure while hexagonal crystal formation occurred at the pressure of 2 kbar. See FIG. 3. Under these same solution conditions but with higher PEG concentrations (9.0-9.5%) at pH 7.0-7.4, formation of both amorphous precipitate and crystals occurred concurrently at 2 kbar.

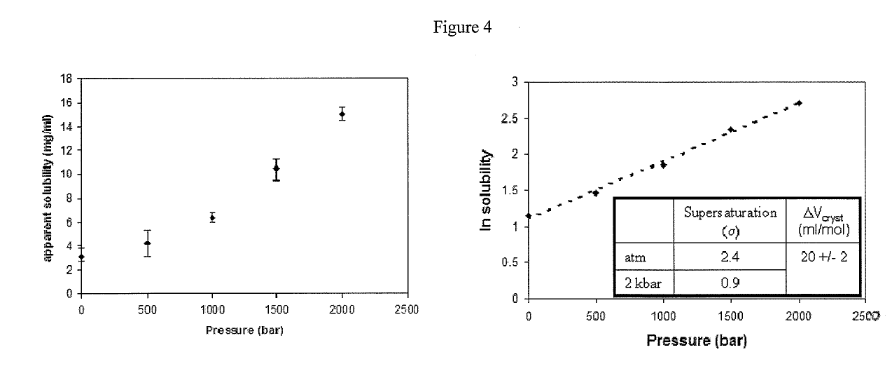

To determine if the pH change of 8.6 to 7.0 was driving the pressure-crystallization phenomenon the solubility of rhGH as a function of pressure at pH 7.0 in the absence of PEG-6000 was determined (FIG. 4). Solution conditions were 100 mM Tris, 500 mM sodium acetate, pH 7.0, 25° C. As can be seen in FIG. 4A, in the absence of PEG-6000 solubility increased with increasing pressure. FIG. 4B shows the graph of natural log of solubility plotted as a function of pressure. The inset table shows the supersaturation and volume change at atmospheric pressure and at 2 kbar pressure.

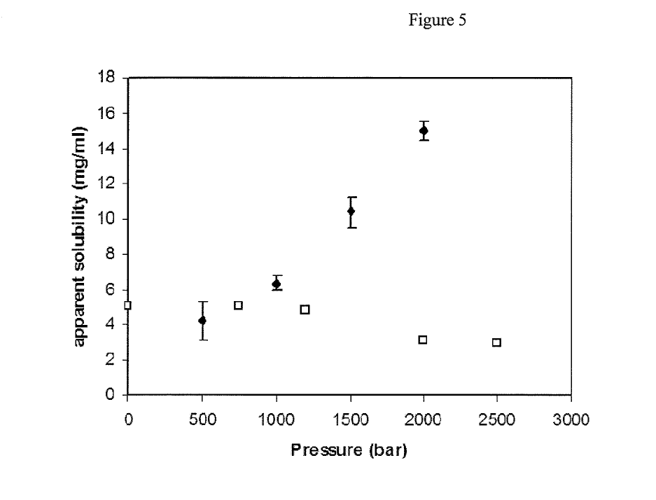

In the presence of 8% PEG-6000, the solubility of rhGH begins to decrease around the pressure of 1.5-2 kbar. FIG. 5 shows the apparent solubility of rhGH as a function of pressure in the absence (closed diamond) and presence of 8% PEG-6000 (open square). Solution conditions were pH 7.0, 100 mM Tris, 500 mM sodium acetate, temperature at 25° C.

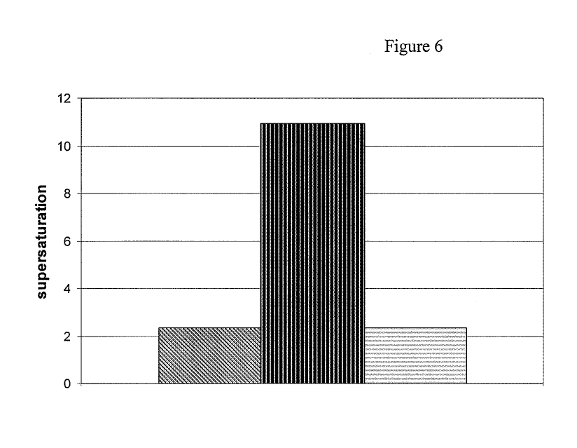

FIG. 6 shows the supersaturation values and crystallization results obtained for rhGH at atmospheric and elevated pressures. The bar with the diagonal stripes shows the supersaturation value of rhGH at elevated pressure; crystals were formed after 12 hours. The bar with vertical stripes shows the supersaturation value of rhGH at atmospheric pressure under the same solution conditions; an amorphous precipitate was formed after 12 hours. The bar with horizontal stripes represents a rhGH solution having the same supersaturation value (obtained by decreasing protein concentration) at atmospheric pressure; rhGH remained in solution.

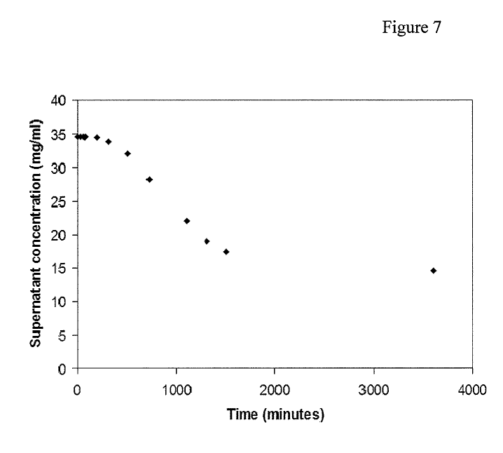

FIG. 7 shows the concentration of rhGH in the supernatant over time at a pressure of 2 kbar during the formation of crystals. Solution conditions are 35 mg/mL rhGH, 100 mM Tris, 8% PEG-6000, 500 mM sodium acetate, pH 7.0, 25° C.

Thus, hexagonal crystals of rhGH can be formed by applying hydrostatic pressures to the protein sample in the presence of a preferential excluding agent such as PEG. In the absence of pressure under the same solution conditions, an amorphous precipitate is formed. The effect of pressure on PEG in solution also teaches that pressure may have a broad-ranging effect on solvent-excluding excipients in general.

### Example 2

### rhGH Production, Crystallization and Crystal Analysis

Cloning, sequence analysis and expression plasmid construction were completed at BaroFold Inc. (Boulder, Colo.). Competent Rosetta DE3 cells containing the pET-21a(+)-rhGH expression plasmids were incubated on LB (Luria-Bertani) agar plates with 50 μg/mL chloramphenicol and ampicillin. Fresh colonies were selected and added to 50 mL of complex media containing 4% yeast extract (Bacto), 1% NaCl, 1% glycerol, 50 μg/mL chloramphenicol and ampicillin and 100 mM MES in a 200 mL baffled flask. Two cultures were placed in a shaker/incubator at 37° C. and 300 rpm and allowed to grow overnight. The cultures (OD₆₀₀=15) were then added to 4% yeast extract, 1% NaCl, 2.5% glycerol, 50 μg/mL chloramphenicol and ampicillin to a final volume of 4 liters in a 4-liter Biostat B (B. Braun Biotech Inc., Allentown, Pa., USA). The culture was induced with 75 μM (final concentration) isopropyl-β-D-thiogalactopyranoside (IPTG) at OD₆₀₀=16 and ampicillin was again added at 50 μg/ml upon induction. Growth continued until OD₆₀₀=30. Ampicillin was again added at. Cells were harvested by centrifugation at 9500 rpm for 10 minutes and the pellets were stored at −20° C.

The rhGH protein was extracted as inclusion bodies from the cell pellets using a Panda 2K high pressure homogenizer (GEA Niro Soavi North America, Bedford, N.H.). Briefly, cells were added to 10% w/v of 10 mM Tris pH 7.5, 1 mM EDTA to a uniform consistency then passed through the Panda 2K operating at 70 MPa. The protein rich inclusion bodies were centrifuged at 9500 rpm for 30 minutes and stored at −20 C.

For refolding and purification, rhGH inclusion bodies were suspended in 5 mL of 8M urea, 20 mM Tris, 20 mM cysteine (pH 8.0) at a protein concentration of 1 mg/mL. After 1 hr of mixing at room temperature, the solution was diluted to a final volume of 40 mL in a refolding buffer of 20 mM Tris, 15% glycerol, 1M urea, 2.5 mM cysteine (pH 8.0) at a protein concentration of 0.125 mg/mL and held at 4° C. overnight.

The refold mixture was clarified by centrifugation and the supernatant loaded onto a 50 mL Toyopearl® Super Q 650M preparative column (Tosoh Bioscience, Stuttgart, Germany) equilibrated in 20 mM Tris (pH 8.0). The rhGH was recovered by elution with a 10-column volume gradient from 0-500 mM NaCl in 40 mM Tris and 0.4 M urea (pH 8.0). Fractions were analyzed using non-reducing SDS-PAGE and fractions enriched with rhGH were pooled. The pooled samples were added to an equal volume solution of 4M NaCl for a final salt concentration of 2 M (25° C.). The salt-rich rhGH solution was loaded onto a 75 mL Phenyl Sepharose™ High Performance (GE Healthcare, Piscataway, N.J., USA) column equilibrated with 20 mM sodium phosphate, 2M NaCl (pH 7.4). The rhGH was recovered by elution with a 5-column volume gradient from 2-0M NaCl in 20 mM sodium phosphate (pH 7.4). Fractions were analyzed using non-reducing SDS-PAGE and fractions enriched with rhGH were pooled and stored at −20° C.

In order to prepare the protein for crystallization at varying pressures, the refolded and purified rhGH was buffer exchanged into the appropriate crystallization buffer (in the absence of PEG-6000) using an Amicon pressurized ultrafiltration cell fitted with a 3,000 MWCO membrane. All batch crystallization solutions were placed in heat-sealed 1-mL syringes (Becton-Dickinson and Co., New Jersey) with excess air removed. The high-pressure samples were placed into a 150-mL high pressure vessel (BaroFold, Inc., Boulder, Colo., USA) and pressure was generated using an automated 400 MPa high-pressure generator (BaroFold, Inc., Boulder, Colo., USA) with water as the pressure transmitting fluid. A 30% stock solution of PEG-6000 was prepared in each crystallization buffer to be mixed with the protein stock solution to obtain the correct PEG and protein final concentrations. The PEG-6000 solution was added to the protein solution to initiate crystallization at varying PEG-6000 and protein concentrations. High pressure samples were immediately pressurized after addition of the PEG-6000 to the protein solution. Crystallization was allowed to proceed for up to 24 hours prior to depressurization and harvesting crystals. Crystals were harvested by vacuum filtration and rinsed twice with 1-volume of 5 mM sodium acetate (pH 5.3). Protein concentration was quantified by A₂₇₈ absorbance using an extinction coefficient for rhGH of 18,890 (cm mol/liter)⁻¹.

rhGH solubility as a function of pressure in Tris-HCl buffer containing 0.5M NaAc was determined by dissolution from an excess of crystals in the appropriate buffer (varying amounts of PEG and pH) at high pressures for 24 hours. Pressure was generated as described above. The solution was centrifuged and the supernatant concentration was determined using UV spectroscopy (Agilent 8453, Santa Clara, Calif.) with an extinction coefficient of 18,890 (cm mol/liter)⁻¹ at 278 nm (St John et al. 2001). The partial molar volume change for crystallization was determined using Eqns. 8-10.

High-pressure light scattering (static (SLS) and dynamic (DLS)) measurements were obtained using static, 90° light scattering with a Brookhaven light scattering system (Brookhaven Instruments Corporation, Holtsville, N.Y.) equipped with a custom-designed high pressure setup. Static light scattering was measured at a wavelength of 633 nm. High pressure samples were placed in a custom-designed quartz cuvette which was then placed in a custom-built, temperature controlled high-pressure vessel surrounded by decalin as the pressure-transmitting fluid. All buffers (excluding PEG) and protein solutions used during light scattering experiments were filtered with a 0.02 micron Anatop 25 syringe filters (Whatman International Ltd.) and the pressurizing fluid was filtered with 0.2 micron Anatop 25 syringe filters (Whatman International Ltd.). Ultra-pure Millipore water (18 milliohm) was filtered with 0.02 micron filters before the addition of PEG. For SLS measurements, light scattering intensity was measured for each protein solution at varying protein concentrations (0.5-5.0 mg/mL) over a pressure range of 0.1-300 MPa. SLS for PEG was over the range of 1-10% w/v PEG. Osmotic virial coefficients as a function of pressure were determined using equation 2, as previously reported (Crisman and Randolph (2009) Biotechnol. Bioeng. 102:483). Dynamic light scattering measurements on rhGH were made at a wavelength of 633 nm at a scattering angle of 90° and the measurement duration for each sample was 5 minutes. All samples were maintained at 25 C. Data analysis was performed using the CONTIN method provided from Brookhaven Instrument Company.

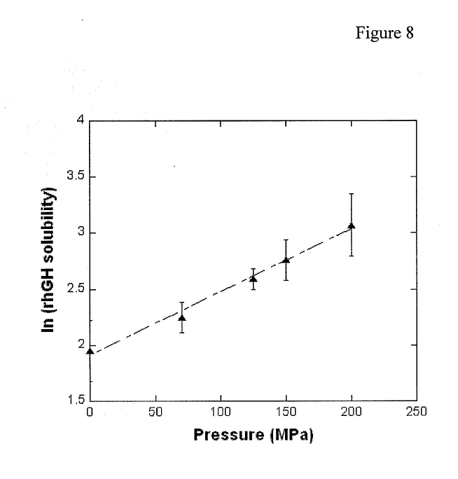

Solubility of rhGH, in the absence of PEG-6000, was measured as a function of pressure. The solubility of rhGH increases from 7.06±0.43 mg/mL at 0.1 MPa to 22.47±2.55 mg/mL at 200 MPa. FIG. 8 is a semi-log plot of the solubility of rhGH as a function of pressure in the absence of PEG-6000. A linear fit to this data, using Equation 10, indicates an exponential increase in solubility with pressure, resulting in a ΔVₓₜₐₗ of 21±2 mL/mol.

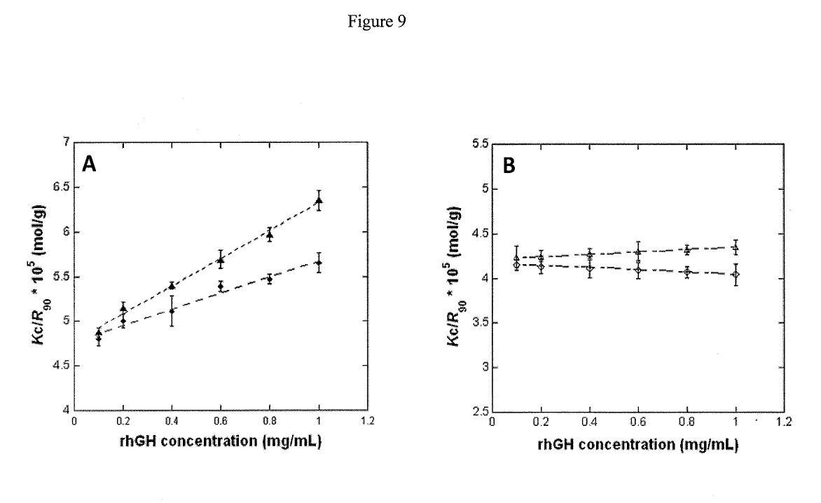

In the absence of PEG-6000, pressure increases the value of B₂₂ from 4.51±0.4* 10⁻⁴ to 8.3±0.3*10⁻⁴ mL mol/g² (FIG. 9). The addition of 6% PEG-6000 results in a negative B₂₂ value for rhGH at atmospheric pressure of −1.0±0.1*10⁻⁵ mL mol/g² whereas pressure increases the B₂₂ value to 1.0±0.1*10⁻⁴ mL mol/g² at a pressure of 250 MPa. Table 1 summarizes the B₂₂ values as a function of PEG-6000 concentration at pressures of 0.1 MPa and 250 MPa.

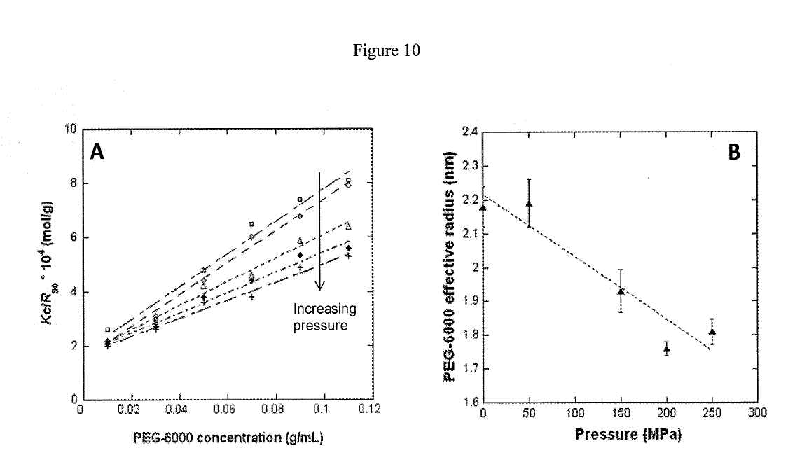

Static light scattering of PEG-6000 as a function of pressure is shown in FIG. 10A. Increasing pressure decreases the PEG virial coefficient (B₃₃) from 3.03±0.35*10⁻³ mL mol g⁻² at 1 MPa to 1.68±0.24*10⁻³ mL mol g⁻² at 250 MPa. The apparent radius (R₃) can be determined from the virial coefficient (Cotts and Selser (1990) Macromolecules 23:2050) using Equation 5. The apparent radius decreases from 2.20±0.06 nm at 1 bar to 1.81±0.03 nm at 250 MPa (FIG. 10B).

The volume fraction of PEG in solution can be determined using equation 12 (Cotts and Selser 1990).

$$φ = \frac{{cN}_{A} [ \frac{4}{3} π {R}_{3}^{3} ]}{{M}_{3}} \qquad ( 12 )$$

The decrease in the effective radius of PEG-6000 going from a pressure of 0.1 MPa to 250 MPa results in a decrease in volume fraction for PEG-6000 of ca. 20%.

TABLE 1
Apparent B₂₂ as a function of PEG-6000 concentration obtained from static
light scattering measurements at pressures of 0.1 MPa and 250 MPa.

| | B₂₂ × 10⁴ | B₂₂ × 10⁴ |
|---|---|---|
| PEG-6000 | (mL mol g⁻²) | (mL mol g⁻²) |
| (% w/v) | P = 0.1 MPa | P = 250 MPa |
| 0 | 4.5 | 8.3 |
| 3 | 1.8 | 4.3 |
| 6 | −0.10 | 1.0 |
| 8 | −1.35 | −0.9 |

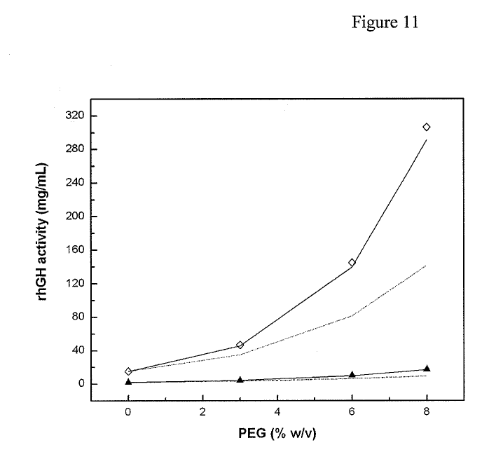

Supersaturated protein solutions are far from being ideal. FIG. 11 shows the protein activity at pressures of 0.1 MPa and 250 MPa at a protein concentration of 15 mg/mL. The dashed lines represent the rhGH activity based on pressure-corrected hard-sphere excluded-volume calculations using R₂ₘᵢₙ and the solid lines represent the hard-sphere approximation using the frictional ratio of 1.21 determined for growth hormone (Conde et al. (2005) Eur. J. Biochem. 32:563).

At a protein concentration of 15 mg/mL rhGH the protein behavior is essentially ideal with an activity coefficient of 1.001. Increasing the PEG-6000 concentration results in an increase in the protein activity from a value of 15 mg/mL at 0% PEG-6000 to a value of 306 mg/mL at 8% PEG-6000 at a pressure of 0.1 MPa. Upon increasing the pressure to 250 MPa, the protein activity decreases to a value of 1.8 mg/mL in 0% PEG and 17.6 mg/mL in 8% PEG.

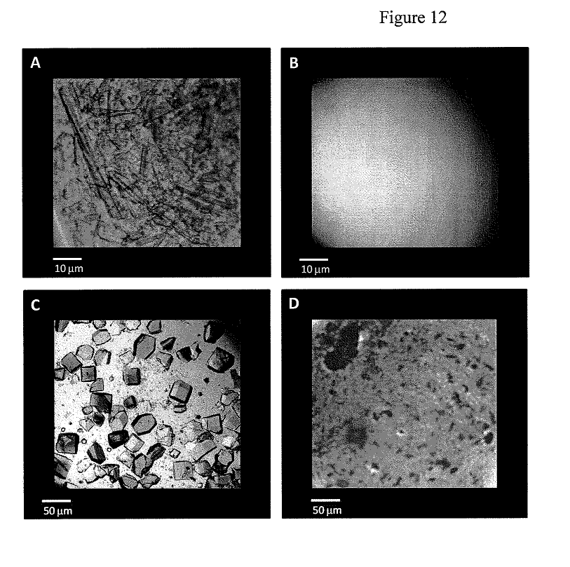

rhGH crystallization at atmospheric pressure occurs at a protein concentration of 15 mg/mL in 6% PEG-6000 (FIG. 12A). Upon pressurization at the same solution conditions, crystallization does not occur (FIG. 12B). The activity at these solution conditions are 144.9 mg/mL and 10 mg/mL at 0.1 MPa and 250 MPa, respectively. rhGH crystallization at 250 MPa occurs at 35 mg/mL in 8% PEG-6000 (FIG. 12C). The same solution condition at atmospheric pressure results in the formation of amorphous precipitate (FIG. 12D). The rhGH activities for these solution conditions are 714 mg/mL and 41.1 mg/mL at 0.1 MPa and 250 MPa, respectively.

Upon pressurization, the protein-solvent system will evolve toward the global conformation that occupies the least volume. For example, dissolution experiments of glucose isomerase crystals, as a function of pressure, resulted in a ΔVₓₜₐₗ of −54±31 mL/mol (Suzuki et al. 2002a; Suzuki et al. 2002b; Kadri et al. (2003) J. Physics=Condenses Matter 15:8253) and crystal formation occurred at an accelerated rate compared to crystal growth at atmospheric pressure (Visuri et al. 1990). However, the increase in solubility of rhGH with increasing pressure, shown in FIG. 8, resulted in a positive ΔVₓₜₐₗ of 21±2 mL/mol. This value (ΔVₓₜₐₗ=−ΔV) resulted in a decrease in the protein chemical potential as a function of pressure Δμ₂^{P}) of −5250 J/mol and high pressure crystallization of rhGH did not occur at protein concentrations up to 50 mg/mL.

The pressure effect on protein solubility has a direct impact on the protein supersaturation and, therefore, on the thermodynamic driving force for protein crystallization. Increases in protein solubility with increasing pressure have been shown to dramatically slow down, or inhibit protein crystallization (Gross and Jaenicke 1991; 1992; Lorber et al. 1996; Webb et al. 1999; Waghmare et al. 2000; Kadri et al. 2003). Our results suggest that increasing pressure decreases the supersaturation of rhGH in solution, decreasing the driving force for spontaneous nucleation and crystal growth.

Several groups have studied the protein intermolecular interactions in the presence of PEG using small angle x-ray scattering and static light scattering (Kulkarni et al. (1999) Physical Review Letters 83:4554; Hitscherich et al. (2000) Protein Sci. 9; Casselyn et al. (2001) Acta Cryst 232; Finet and Tardieu (2001) J. Cryst. Growth 232; Tanaka and Ataka (2002) J. Chem. Phys. 117:3504). These results indicated that PEG is a useful crystallization cosolute because it can effectively induce attraction between protein molecules via steric exclusion of PEG from the rhGH domain. This thermodynamically unfavorable preferential exclusion between PEG and protein results in an increase in protein activity leading to the action of PEG as a protein precipitant. In agreement, our results (FIG. 11) show that increasing PEG-6000 concentration increases the rhGH activity and leads to protein precipitation (either as a crystal or an amorphous precipitate) at varying PEG concentrations and pressures.

Macromolecules in water-based solutions have been known to change secondary and tertiary structure at elevated pressures (Taniguchi and Takeda (1992) Proceedings of the First European Seminar of High Pressure Biotechnology). Pressure is known to reduce the degree of hydrogen bonding in water and diminish the strength of the hydrophobic interactions (Tanaka et al. (1974) J. Colloid Interface Sci. 46; Nishikido et al. (1980) J. Colloid Interface Sci. 46:474). Interestingly, the two types of interactions that control PEG solubility are hydrogen bonding and hydrophobic interactions (Cook et al. (1992) Physical Review Letters 69) suggesting that high pressure can perturb the solution conditions of PEG in water. In agreement, our results suggest that increasing pressure perturbs the PEG-water system as seen in a decrease in B₃₃. Prior results (Devanand and Selser (1991) Macromolecules 24:5943; Cook et al. 1992) have attributed the B₃₃ value with strong water-PEO interactions through hydrogen bonding suggesting that the decrease in B₃₃ with increasing pressure (FIG. 10A) could be a result of the reduction of PEG-water hydrogen bonding at elevated pressures. This decrease in hydration resulted in a decrease in the B₃₃ apparent radius of PEG-6000 from 2.2±0.06 nm at 0.1 MPa to 1.8±0.04 nm at 250 MPa.

The decrease in apparent radius of PEG at elevated pressure decreases the excluded volume of PEG in solution by ca. 20% at a pressure of 250 MPa. For protein crystallization with PEG as the precipitating agent, this decrease in excluded volume of PEG at elevated pressures results in a decrease in the depletion interactions between protein molecules, which are dependent on the size and concentration of PEG. Our results suggest a decrease in apparent radius of PEG upon pressurization results in a decrease in attractive depletion interactions between the protein molecules. Thus, it is possible to vary the strength of an attractive potential between protein particles in the presence of PEG by simply changing the hydrostatic pressure on the solution.

At atmospheric pressure, rhGH crystal growth occurs in the presence of 6% PEG-6000 at a protein concentration of 15 mg/mL. The protein activity of this solution is 144 mg/mL. Upon pressurization to 250 MPa, the activity decreases to a value of 10.0 mg/mL and crystallization does not occur.

For the hard-sphere approximation, the choice of the most compact spherical configuration of the protein, R₂ₘᵢₙ is not a bad assumption at the given solution conditions. The high salt concentration shields the charge-charge repulsion resulting in a decrease in the effective hydrated radius. However, the discrepancy between this hard-sphere activity and the experimentally determined atmospheric activity at higher PEG concentrations is not surprising. The hard-sphere approximation used for rhGH was a minimum value that did not take in to account the extent to which the protein was solvated. In addition, we are neglecting higher order excluded terms in our hard-sphere assumption which should become increasingly important at the higher PEG concentrations. However, upon correcting the hard-sphere approximation using the protein friction factor of 1.21 results in a very good fit of the hard-sphere excluded volume approximation with the experimental data.

Upon increasing the protein concentration to 35 mg/mL, the formation of amorphous precipitate of rhGH occurred at atmospheric pressure in solution conditions containing 8% PEG-6000 and a protein concentration of 35 mg/mL whereas crystallization occurred at 250 MPa. These solution conditions resulted in a protein activity of 714 mg/mL and 41.1 mg/mL at 0.1 and 250 MPa, respectively. These results show that high pressure can be successfully used to modify the system thermodynamics to obtain solution conditions that favor crystallization over amorphous precipitate.

The steric exclusion of PEG from the protein domain results in a thermodynamically unfavorable preferential exclusion interaction that dominates the protein crystallization and precipitation, independent of pressure. The range of these interactions is given by the protein and polymer apparent size. Thus, it should be possible to vary the range (by varying the polymer size) and the strength (by varying the polymer concentration) of the volume exclusion interactions for a given system. Our results suggest that the decrease in the thermodynamic instability with increasing pressure decreases the unfavorable preferential exclusion interactions and requires more PEG and/or protein for crystallization at elevated pressures.

Crystallization of rhGH occurred at 250 MPa in the presence of 8% PEG-6000 while amorphous precipitate formed at 0.1 MPa in the same solution conditions. The addition of PEG to the protein solution increases the effective concentration of the protein solution, as seen with the increase in protein activity. Increasing pressure was shown to decrease the volume exclusion interactions, requiring higher concentrations of both PEG-6000 and rhGH for crystallization at high pressure. The formation of protein crystals, not amorphous precipitate, occurs at elevated pressures due to the pressure effects on the colloidal interactions which reduce the short-range contacts driving protein precipitation, allowing for increased supersaturation without increasing the rate of aggregation. In the presence of a preferential excluder such as PEG, pressure provides a novel technique for adjusting protein interactions leading to crystallization.

### Example 3

### Production of Xylanase Crystals

PEG-6000, magnesium chloride (MgCl₂) and tris(hydroxymethyl) aminomethane (Tris) were purchased from Sigma-Aldrich (Sigma-Aldrich, St. Louis, Mo.). Xylanase was supplied in a purified form at a concentration of 36 mg/mL in 0.18 M sodium/potassium phosphate buffer (pH 7.0) and 43% (w/v) glycerol (Hampton Research Inc., Aliso Viejo, Calif.). The supplied xylanase buffer was exchanged to 10 mM Tris-HCl (pH 7.5) with 5-10 dilution/concentration cycles using a 5 kDa membrane cutoff Amicon Ultra-4 centrifugal filter device (Millipore Corp., Bedford, Mass.). The final protein concentration was determined to be 28-32 mg/mL. All solutions were filtered with a 0.22 μm Millex GV filter unit (Millipore Corp.) with the exception of PEG-6000. The samples were loaded into 1 mL BD syringes with heat sealed tips and the plungers re-inserted.

Pressure was generated with an instrument as described in Example 1. Crystallization screens using the hanging-drop vapor diffusion method revealed xylanase crystals could be grown at room temperature in 16 hours with a broad range of PEG-6000 and protein concentrations in a solution containing 50 mM Tris-HCl (pH 8.5) and 100 mM MgCl₂. Batch crystallization studies of xylanase at atmospheric pressure were performed in 1 mL BD syringes at 25° C. in the presence of PEG-6000. The samples were visually analyzed using a stereomaster zoom microscope equipped with a digital microscope head (MZD, Fisher Scientific) and polarization kit (Westover Scientific). Solubility data were determined by centrifuging the batch sample (Eppendorf centrifuge, 13,000 rpm for 3-4 minutes) and analyzing the protein concentration in the supernatant by UV-spectrophotometry at 280 nm.

In addition to the atmospheric crystallization optimization studies, a broad pressure crystallization screen was performed. Batch xylanase samples (100 μL) were placed at pressures of 1, 1000, 1500, 2000, and 2500 bar, with xylanase concentrations of 12.5-20 mg/mL and 13.5-25% PEG-6000 with 50 mM Tris-HCl (pH 8.5) and 100 mM MgCl₂ for approximately 3 days (66 hrs). Additional pressure screens were done in the absence of PEG in an identical fashion.

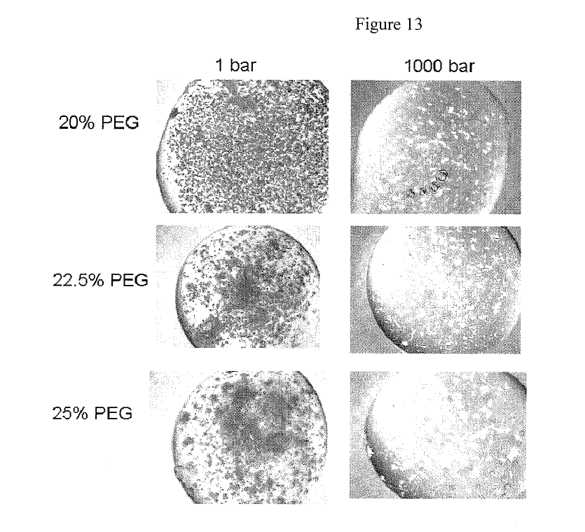

Xylanase (13.5 mg/mL) in the presence of varying concentrations of PEG-6000 (20%, 22.5% and 25%) formed amorphous precipitate at atmospheric pressure while samples placed under the pressure of 1000 bar formed crystals and no visible amorphous precipitate. These results are shown in FIG. 13.

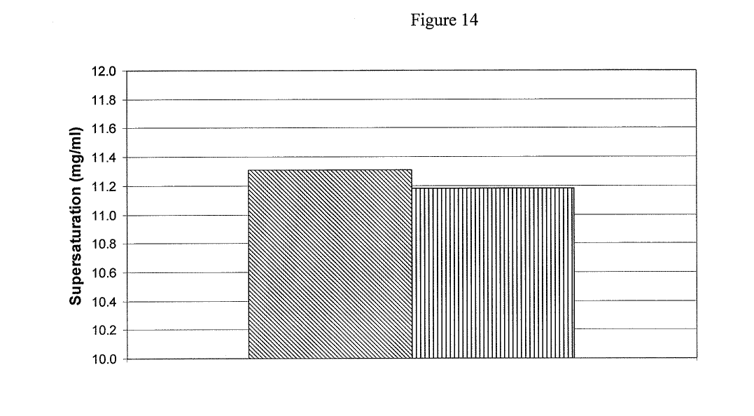

FIG. 14 shows the supersaturation values and crystallization results for xylanase samples at atmospheric pressure (diagonal stripes) and at the pressure of 1000 bar (vertical stripes). The sample at atmospheric pressure produced amorphous precipitate at this supersaturation value while the pressure treated sample formed crystals. Even though the supersaturation values of xylanase at 1 bar and 1000 bar were nearly identical in the same solution conditions, the atmospheric sample formed amorphous precipitate while the sample treated with pressure formed crystals. These results suggest that there may be a pressure effect on the PEG-PEG or PEG-protein interactions that promotes protein crystallization and not simply a phenomenon where pressure is driving the supersaturation value into the “crystallization window”.

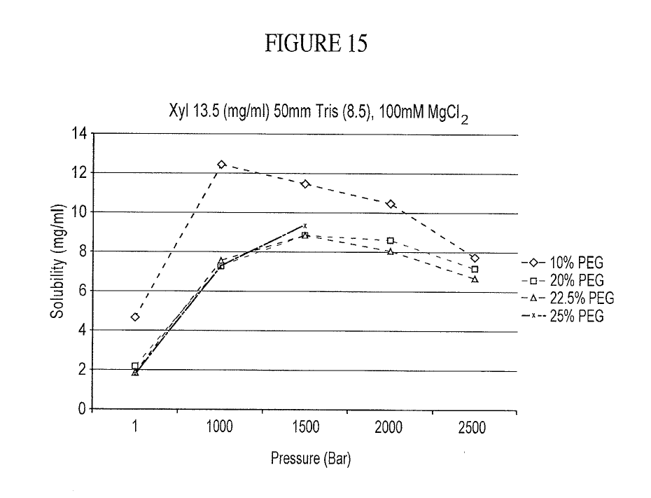

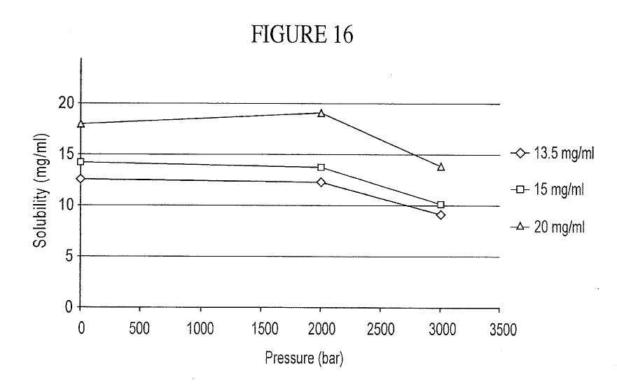

FIG. 15 shows the solubility of xylanase as a function of pressure in the presence of varying amounts of PEG-6000. These data suggest that solubility of xylanase first increases with increasing pressure (up to 1000 bar in the presence of 10% PEG-6000 and up to 1500 bar in the presence of 20%, 22.5% and 25% PEG-6000) and then unexpectedly begins to decrease. Furthermore, this change in solubility as a function of pressure in the presence of PEG-6000 occurs at a different pressure point than in the absence of PEG. FIG. 16 shows the decrease in xylanase solubility as a function of pressure in the absence of PEG-6000. This result suggests that above 2000 bar the decrease in solubility as a function of pressure could be protein specific, possibly due to changes in the conformational and/or colloidal stability of the protein. Accordingly, a majority of the crystallization experiments described in the document were performed between 1000 and 1500 bar to avoid conformation changes in the native protein structure.

Concurring with Example 1, these results indicate that the combination of high pressure and the presence of PEG in solution promotes protein crystallization.

### Example 4

### The Pressure Effect on Amorphous Precipitate and Crystal Mixture Formed at Atmospheric Pressure

A solution of Xylanase (13.5 mg/mL) in 50 mM Tris-HCl, pH 8.5, 100 mM MgCl₂, 10% PEG-6000 was allowed to sit in batch mode overnight (approximately 16 hours) at atmospheric pressure. Visual inspection indicated that a mixture of amorphous precipitate and crystals were present the next day. This mixture was next treated with varying pressures from 1-3 kbar for 24 hours or 66 hours. The samples were visually analyzed using a stereomaster zoom microscope equipped with a digital microscope head (MZD, Fisher Scientific) and polarization kit (Westover Scientific). Xylanase solubility was measured by the absorption at 280 nm of the supernatant following clarification by centrifugation.

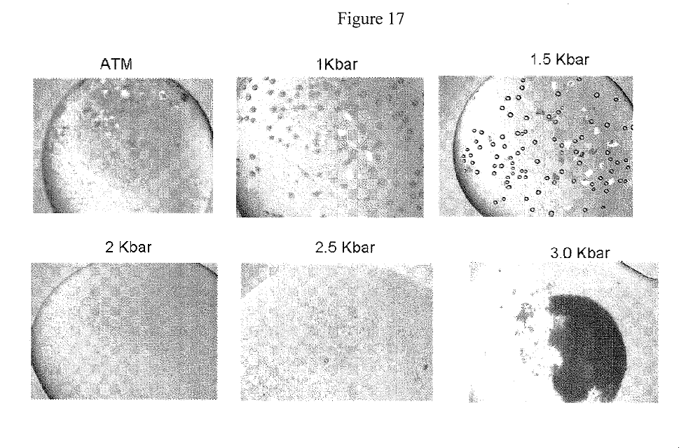

FIG. 17 shows that upon pressurization to 1-1.5 kbar the amorphous precipitate disaggregates while the crystals remain in solution. The 1 kbar treated sample was the most homogeneous with no detectable levels of precipitate present. This observation was not unexpected and agrees with the solubility data shown in FIG. 15 for xylanase in the presence of 10% PEG-6000. Pressurization to 2 kbar resulted in the exclusive formation of amorphous precipitate. The decrease in solubility shown in FIG. 15 and FIG. 16 suggests that above 2 kbar the supersaturation of the protein in solution is too high and thus favors formation of amorphous precipitate. The removal of an amorphous precipitate from a mixture containing crystals and amorphous precipitate with high pressure has not been reported previously.

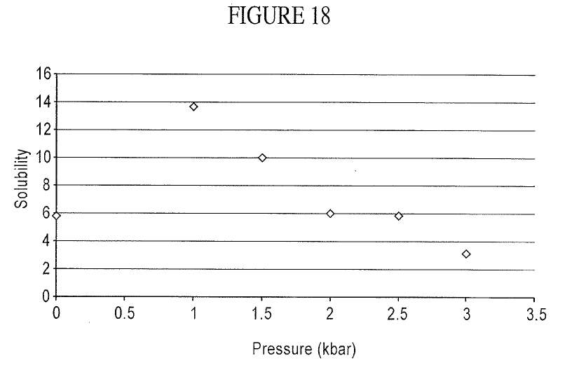

FIG. 18 shows the concentration of xylanase in the supernatant as a function of pressure. The sample treated with 1 kbar of pressure had the highest amount of soluble protein. This result suggests that at 1 kbar, the amorphous precipitate is being renatured while under pressure. With the ability to control the pressurization and depressurization rates it should also be possible to precisely modulate crystal growth when starting with a mixture of amorphous precipitate and crystals.

### Example 5

### Crystallization Kinetics at Elevated Pressure

The pressure effect on the batch crystallization kinetics of rhGH was studied. The results show that the mass crystal growth rate increases from 0.42±0.11 mg mL⁻¹ hr⁻¹ at 0.1 MPa to 1.0±0.10 mg mL⁻¹ hr⁻¹. In an attempt to separate nucleation and growth rate on the overall crystallization process, particle counting and sizing data was obtained to give nucleation rates of 4.6±0.7×10³ # mL⁻¹ hr⁻¹ and 2.9±1.0×10³ # mL⁻¹ hr⁻¹ at 0.1 MPa and 250 MPa, respectively and growth rate constants, assuming a first order surface integration process, of 0.73±0.14×10⁴ cm hr⁻¹ at 0.1 MPa to 2.1±0.2×10⁻⁴ cm hr⁻¹ at 250 MPa, respectively. In addition, the scalability of high pressure protein crystallization of rhGH was shown with successful crystallization occurring at a 100-fold increase in volume for the batch crystallization process.

The advancements in crystallization research and development have led to a dramatic growth in the number of proteins that can be isolated and three-dimensional structure determined by x-ray crystallography. Despite the multitude of high-throughput techniques to obtain a single, large crystal for structural determination, little success has occurred for large scale protein crystallization (Basu et al. 2004). The lack of successful industrial protein crystallization can be attributed to the differences in the overall goal for crystallization: the goal for X-ray quality crystals is a small number of crystals with good size and internal quality whereas a good industrial produced pharmaceutical crystal should have sufficient physical robustness, chemical and biological stability and scalability (Jen and Merkle (2001) Pharm. Res. 18:1483).

Industrial crystallization of small molecule pharmaceuticals has been successfully used for decades (Hancock and Zografi 1997). Numerous advantages of the crystalline product include rapid purification, better handling, improved stability and the possibility of controlled release (Hallas-Moller et al. 1952; Jen and Merkle 2001; Margolin and Navia 2001; Shenoy et al. (2001) Biotechnol. Bioeng. 73:335; Basu et al. 2004). It is thought that these characteristics will also be possessed by protein crystals. In addition, protein crystals may provide a novel method to achieve high concentration, low viscosity antibody preparations for delivery of large protein doses in a small volume (Yang et al. 2003; Basu et al. 2004).

In spite of the potential advantages of protein crystal product, the knowledge about how to crystallize proteins at a large scale in a production process has found limited use in the industry (Estell (2006) National Academy of Engineering 36). One of the major drawbacks to scalability is the complex nature of protein molecules, making their crystallization a difficult and challenging process in that each protein to be crystallized is unique, and the development of a large-scale crystallization process must be based on experimental data. Thus, a significant amount of protein will be used on the small-scale batch crystallization studies before scale-up can be attempted in hopes of producing a safe, stable and efficacious formulation that can be delivered in a patient-friendly manner. Although there are a handful of proteins being crystallized commercially (glucose isomerase (Visuri 1992), insulin (Eli Lilly), amylase (Miles) and lipase (Novo), large scale crystallization is rarely practiced due to the lack of large amounts of protein and the lack of scalability of the process (Saikumar et al. (1998) J. Cryst. Growth 187:277).

One of the major challenges to industrial protein crystallization is the ability to generate conditions at the large scale that are similar to those at the small scale. Diffusion limitations in large-scale crystallization processes require precise control of flow-rates, mixing and agitation such that the supersaturation levels both locally and globally are controlled by minimizing heat and mass transfer limitations (Harrison et al. (2003) Bioseparations Science and Engineering. Oxford University Press, New York.). A change in high pressure, however, is transmitted through aqueous solutions nearly instantaneously without the need of controlled mixing. In addition, pressure is known to affect the protein solubility and hence, protein crystallization (Webb et al. 1999; Waghmare et al. 2000; Suzuki et al. 2002a; Nagatoshi et al. (2003) J. Cryst. Growth 254:188). The first report on protein crystal growth under pressure revealed that the yields of small glucose isomerase crystals could be enhanced with increasing pressure (Visuri et al. 1990). In contrast, several high pressure crystallization studies with lysozyme reported that the solubility increased and the growth rate of the crystal and the nucleation rate decreased with increasing pressure (Suzuki et al., 1994; Schall et al., 1994; Saikumar et al., 1995; Lorber et al., 1996; Takano et al., 1997; Suzuki et al., 2002). Similar observations of high pressure inhibition of crystal growth were also reported with subtilisin (Webb et al., 1999; Waghmare et al., 2000a; Waghmare et al., 2000b). The variable effect of high pressure on protein crystallization, despite the potential benefits of high pressure industrial processing, suggests a lack of understanding of the pressure effects on the crystallization process.

This example elucidates the pressure effects on the overall crystallization kinetics for rhGH at pressures of 0.1 MPa and 250 MPa. An increase in crystallization rate at elevated pressures is likely due to an increase in the growth rate constant due to an increase in surface diffusion. In addition, the ability of high pressure to act on the whole system instantaneously and uniformly should provide a scalable process for producing protein crystals on the industrial scale.

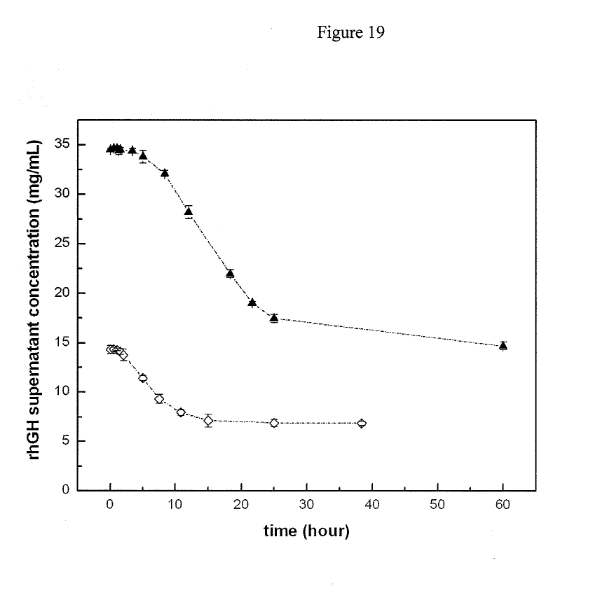

rhGH production, refolding, purification and crystallization are performed as disclose din Example 2. FIG. 19 shows the rhGH solubility versus time for unseeded, batch crystal growth at 0.1 and 250 MPa. Residual protein concentration during crystal growth was determined by A₂₇₈ absorbance using an extinction coefficient for rhGH of 18,890 (cm mol/liter)⁻¹. From the slope of the linear portion of the data the overall crystal growth at a pressure of 0.1 MPa was determined to be 0.42±0.11 mg mL⁻¹ h⁻¹ compared to 1.01±0.10 mg mL⁻¹ h¹ at 250 MPa. The resulting increase in crystallization rate at elevated pressure relative to the rate at atmospheric pressure could be due to the following factors:
(i) Difference in growth mechanism or surface area for the mass transfer on to the growing face(s) at elevated pressure
(ii) faster primary and/or secondary nucleation
(iii) increase in the growth rate constant

Crystal growth rates have been extensively determined following the growth of a single edge under a microscope. This represents drawbacks when trying to compare these growth rates with those from a batch crystallization process. The growth of a single crystal face is unlikely to represent the mean growth of all faces (Garside et al. (2002) Measurement of Crystal Growth and Nucleation Rates. 2^{nd} Ed. Institution of Chemical Engineers, Rugby, UK). With that said, differences in mean crystal growth determined from a batch processes could arise from differences in the growth mechanism i.e. growth on a single crystal face resulting in rod-like morphology could have a different crystallization rate compared to growth on numerous faces resulting in more symmetric crystal growth (e.g. hexagonal morphology).

In addition, the rate of mass transfer, \dot{m}, from solute to the crystal phase is related to the surface area of the protein (sₚ) (Eqn. 1) (McCabe et al. (2001) Unit Operations for Chemical Engineers, 6^{th} Ed. McGraw-Hill Chemical Engineering Series).

\dot{m}=sₚk_{y}(y−y′) (1)

where, k_{y} is the mass transfer coefficient and (y-y′) is the concentration driving force for mass transfer. An increase in surface area would result in an increase in the rate of mass transfer to the crystal surface potentially resulting in an increase in crystallization rate. Therefore, the morphology of the crystals grown at pressures of 0.1 MPa and 250 MPa would be needed to determine if the differences in crystallization rates are due to the differences in growth rate on the crystallographic faces.

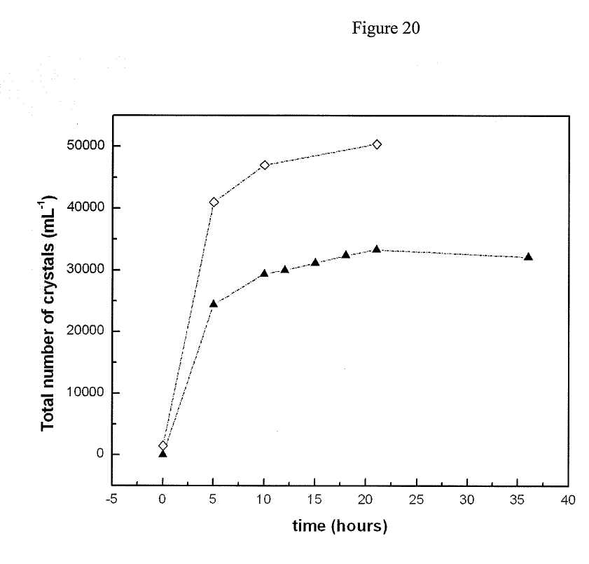

Another possibility for the increased crystallization rate involves the primary nucleation. Primary nucleation can be determined from the total number of crystals over time (Eqn. 2).

$$B = \frac{d N}{d t} \qquad ( 2 )$$

where B is the number of nuclei formed per unit volume per unit time and N is the number of nuclei per unit volume. FIG. 20 shows the total number of crystals per milliliter (N) as a function of time. The nucleation rate, determined from the slope of the line of the initial data points, results in a value of Bₐₜₘ=4.5±0.7×10³ #/(mL-hr) and Bₕₚ=2.9±1.0×10³ #/(mL-hr). The supersaturation ratio, (c-s)/s, where c is the protein concentration in solution and s is the protein solubility at a given solution condition, is 3.4 and 1.8 at 0.1 MPa and 250 MPa, respectively. This result suggests that the increase in the number of crystals at atmospheric pressure, relative to the pressure of 250 MPa, could be due to the increase in the supersaturation ratio of the protein in solution. In addition, these results suggest that the batch crystallization processes at both pressures lead to a limited number of crystals occurring during nucleation, in agreement with the Balanced Nucleation and Growth Model (BNG). However, these results do not provide a mechanism for the increase in crystallization rate at elevated pressure.

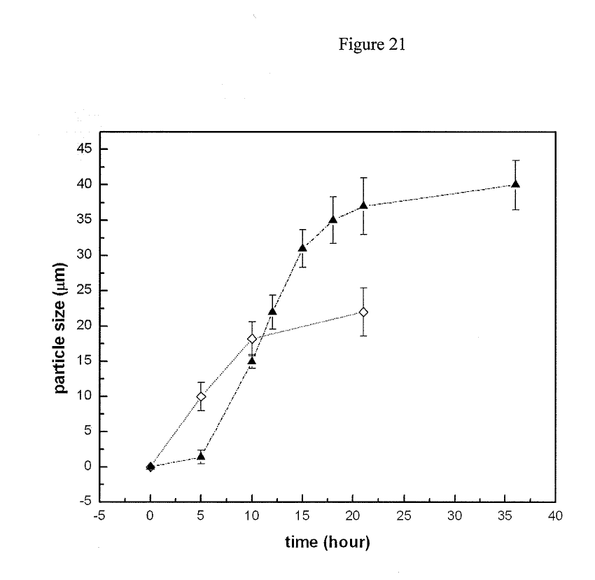

The increase in the overall number of crystals during nucleation at 0.1 MPa resulted in a larger number of smaller crystals compared to those grown at atmospheric pressure (FIG. 21). The mean crystal size determined from particle sizing data at 0.1 MPa is 22±4 μm whereas the crystals grown at a pressure of 250 MPa are 40±5 μm. Therefore, the overall increase in crystallization rate at elevated pressure may be due to the increase in the growth rate of the protein crystals at elevated pressure.

The overall process of crystal growth consists of mass transfer through the crystal boundary layer followed by the surface integration. Assuming these two processes occur in series (Jancic and Grootscholten (1984) Industrial Crystallization. Springer, pp. 434), the overall crystal growth rate can be expressed in terms of the overall driving force,

$$\frac{c - s}{s} ,$$

and the overall crystal growth rate coefficient k_{g}. The growth rate, dL/dt, is a function of supersaturation and can be represented as having a power-law dependence on supersaturation (Eqn. 3) (Jancic and Grootscholten 1984):

$$\frac{d L}{d t} = {{k}_{g} ( \frac{c - s}{s} )}^{b} \qquad ( 3 )$$

where L is the total length of the crystal. The growth rate of most crystals has been shown to be linear with supersaturation (McCabe et al. 2001) therefore, the growth rate can be assumed to be first-order in the surface integration process (b=1). Therefore, integration of the growth rate as a function of supersaturation (determined from FIG. 6-1) resulted in an increase in the growth rate constant, k_{g}, from 0.73×10⁻⁴ cm/hr at 0.1 MPa to 2.1×10⁻⁴ cm/hr at 250 MPa. The growth rate constant is independent of the size of the crystal and therefore may give insight in to the increased growth rate at elevated pressure. Table 2 summarizes the crystallization kinetic parameters as a function of pressure.

The measured protein concentration dependence of the crystal growth rates may provide a clue in to the pressure effects on the underlying growth mechanism by which molecules build up the crystal (Sleutel et al. (2008) Crystal Growth and Design 8:1173). The adsorption of proteins at the solute-crystal interface has been shown to be a complex process involving solute transport from the bulk solution to the surface of the protein, adsorption on to the surface of the protein and spreading of the growth layers on the surface (Weaver and Pitt (1992) Biomaterials 13:577). Determining the rate-limiting step for crystal growth as a function of pressure is important to understand the pressure effects on the molecular pathway a molecule will follow from solution to the crystal.

TABLE 2
Kinetic parameters of crystal nucleation and growth
as a function of pressure.

| | Crystallization | Nucleation | Growth rate |
|---|---|---|---|
| Pressure | rate, R_{c} | rate, B° | constant, k_{g} |
| (MPa) | (mg mL⁻¹ hr⁻¹) | (# mL⁻¹ hr⁻¹) | (cm hr⁻¹) |
| 0.1 | 0.42 ± 0.11 | 4.6 ± 0.7 × 10³ | 0.73 ± 0.14 × 10⁻⁴ |
| 250 | 1.0 ± 0.10 | 2.9 ± 1.0 × 10³ | 2.1 ± 0.16 × 10⁻⁴ |

The error is determined from the best fit line to the corresponding data.

The diffusion controlled transport of a molecule from the bulk to the crystal surface can be discussed using Fick's First Law (Eqn. 4):

$$j = D \frac{\partial c}{\partial x} \qquad ( 4 )$$

where j is the diffusion flux (mol/m²-s), D is the diffusion coefficient (m²/s), c is the concentration (mol/m³) and x is the position (length) (m). The Stokes-Einstein equation can be used to link the diffusion coefficient (D) and the mobility of the particle using Equation 5.

$$j = D \frac{\partial c}{\partial x} \qquad ( 5 )$$

Where k_{b} is Boltzmann's constant, T is the absolute temperature, η is the viscosity of the medium and r is the radius of a spherical particle. Therefore, if the nearly 3-fold increase in growth rate constant at elevated pressure were due strictly to bulk diffusion, it would result from an increase in the diffusion coefficient with increasing pressure. At 2500 bar, the viscosity of the medium decreases by a value of ca. 15% (NIST/ASME Steam Properties Database Version 2.21). Although this decrease in viscosity with increasing pressure may partially describe the increase in growth rate constant at elevated pressure, it does not describe the overall 3-fold increase in the growth rate constant at elevated pressure.

The sticking coefficient, φ, is a measure of the affinity of the protein for the surface of the crystal (Weaver and Pitt 1992). The sticking coefficient is the fraction of the collisions between the protein and an available surface site that result in adsorption and plays a direct role in the rate of adsorption of protein on to the surface of a crystal. Diffusion-controlled adsorption of protein from non-flowing solution has often been modeled using Equation 6 (Young et al. 1988):

$${c}_{s} = 2 {{c}_{B} ( \frac{Dt}{π} )}^{1 / 2} \qquad ( 6 )$$

Where cₛ is the concentration of adsorbed protein, c_{b} is the initial (bulk) protein concentration and D is the diffusion coefficient. The rate of adsorption (R_{A}) can then be defined using equation 7 (Collins and Kimball (1949) J. Colloidal Sci. 4:425):

$${R}_{A} = \frac{d {c}_{s}}{d t} = {( {qv}_{a} {φc}_{B} )}_{j = 0} \qquad ( 7 )$$

Where q=0.5 is the probability, from an unbiased random walk model, that a particle will step to the left and vₐ is defined to be the average jump frequency. Therefore, we can see from Equation 7 that the rate of adsorption is dependent not only on the diffusion coefficient but also on the sticking coefficient.

The protein sticking coefficient is a function of the molecular interactions between the protein and the surface. It is a function of energy barriers, hydrophobic interactions, steric hindrance, electrostatic repulsion, the strength of the protein-surface interaction and other factors which affect the probability of adsorption (Weaver and Pitt 1992). High pressures are known to affect weak interactions for protein stability by decreasing the hydrophobic effect and increasing electrostriction (Balny (2004) J. Phys. Condens. Matter 16:S1245). Therefore, the pressure effects on these weak interactions have the potential to play a significant role on the protein sticking coefficient and hence, the crystal growth rate constant at elevated pressure.

Once the protein is adsorbed to the surface, it must find the correct growth site by diffusing on the surface of the protein. The molecular interactions involved during the surface diffusion depend greatly on the specific interactions of the molecule on the crystal surface (Petsev et al. (2002) PNAS 100:792). Therefore, an activation barrier of correct attachment to the growth site exists that may be of electrostatic origin (Eyring et al. (1980) Basic Chemical Kinetics. Wiley, New York), repulsive potential due to hydrophobic and hydrophilic interactions (Israelachvili (1992) Intermolecular & Surface Forces. Academic Press, San Diego, Calif.) or from the need to expel the water molecules attached to the surface-diffusing molecules and to the growth site (Petsev and Vekilov (2000) Physical Review Letters 84:1339). Therefore, the increase in growth rate constant at elevated pressure could be due to the pressure effects on the surface interactions during surface diffusion to the growth site.

The weak interactions that dominate crystal growth (McPherson (1999) Crystallization of Biological Macromolecules. Cold Springs Harbor, N.Y.) are the same interactions that have significant pressure effects (electrostatic and hydrophobic interactions) (Balny 2004b) on protein stability. It is not surprising, then, that high pressures have shown to have a dramatic impact on crystal nucleation and growth rates (Suzuki et al., 1994; Schall et al., 1994; Saikumar et al., 1995; Lorber et al., 1996; Takano et al., 1997; Suzuki et al., 2002; Waghmare et al., 2000a; Waghmare et al., 2000b). The ability for the diffusing molecule to interact with the surface of the protein, independent of the hydrophobic/hydrophilic repulsion at elevated pressures, has a direct impact on the sticking coefficient. Therefore, the increase in the growth rate constant at elevated pressure could be due to the decrease in the hydrophobic effect and increase in electrostriction with increasing pressure, allowing for favorable surface interactions and diffusion leading to an increase in crystallization rate for rhGH at elevated pressure.

Finally, the ability for pressure to act nearly instantaneously and uniformly throughout the solution is extremely beneficial for producing large scale crystallization batches. The ability to produce rhGH crystals at a 100× scale-up without changes in particle size distribution, in addition to the widely available large-scale pressure equipment, provides an opportunity for industrial production of a protein therapeutic in addition to advantages in industrial crystallization in general. The application of non-batch, solution exchange methodology to protein crystallization under pressure further supports the ability for large scale crystallization.

The results show that the increase in crystallization kinetics of rhGH at elevated pressure could be due to the decrease in the repulsive potentials during adsorption and surface diffusion. Upon increasing pressure, the wetting of the hydrophobic surfaces can decrease the repulsive hydrophilic/hydrophobic interactions allowing for more surface contacts to take place. Once the protein is adsorbed to the surface of the crystal, it must diffuse on the surface to the correct growth site. Bulk diffusion is likely not the rate-limiting step and that the pressure effect on the sticking coefficient and surface diffusion upon pressurization allows for faster growth compared to atmospheric conditions. Lastly, it was possible to scale the high pressure batch crystallization of rhGH 100-fold indicating the usefulness of pressure processing on the industrial scale.

Unless otherwise indicated, all parts and percentages are by weight and all molecular weights are weight average molecular weights. The foregoing detailed description has been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of forming crystals of a molecule in a solution, comprising:
(a) adding a precipitating agent to the solution,
(b) applying to the solution a hydrostatic pressure to form crystals of the molecule, and
(c) depressurizing the solution.

2. The method of claim 1, wherein the crystals are not formed when the solution is at atmospheric pressure.

3. The method of claim 1, wherein the crystals are not formed when the solution is at atmospheric pressure and the conditions of the solution are changed so that the supersaturation value of the solution remains constant.

4. The method of claim 1, wherein an amorphous precipitate is formed when the solution is at atmospheric pressure.

5. The method of claim 1, wherein the morphology of the crystals formed when the solution is subjected to hydrostatic pressure is different from the morphology of the crystals formed when the solution is at atmospheric pressure.

6. The method of claim 1, wherein the number of crystals formed when the solution is subjected to hydrostatic pressure is greater than the number of crystals formed when the solution is at atmospheric pressure.

7. The method of claim 1, wherein the crystals formed when the solution is subjected to hydrostatic pressure are bigger in size than the crystals formed when the solution is at atmospheric pressure.

8. The method of claim 1, wherein the population of crystals formed when the solution is subjected to hydrostatic pressure is more homogeneous than the population of crystals formed when the solution is at atmospheric pressure.

9. The method of claim 1, wherein the amount of any amorphous precipitate formed is less than about 25% of the amount of the crystals.

10. The method of claim 1, further comprising the step of recovering the crystals.

11. The method of claim 1, wherein the molecule is selected from the group consisting of proteins, DNA, RNA, carbohydrates, peptides and polymers.

12. The method of claim 11, wherein the protein is selected from the group consisting of antibodies, Fab fragments, trophic factors, cytokines, lymphokines, toxoids, growth factors, hormones, human growth hormones, growth hormone family members, nerve growth hormones, fertility hormones, postridical hormones, fusion proteins, glycoproteins, synthetic antigens, recombinant antigens, histocompatibility antigens, viral surface proteins, bone morphogenic proteins, enzymes, blood clotting factors, adhesion molecules, multidrug resistance proteins, interleukins, interleukin receptors, chemokines, interferon receptors, T-cell receptors, blood factors, leukocyte markers, monocyte-macrophage colony stimulating factors, granulocyte colony stimulating factors, integrins, selectins, and lectins.

13. The method of claim 11, wherein the protein is selected from the group consisting of erythropoietin, Factor VIII, insulin, amylin, TPA, dornase-α, α-1-antitrypsin, urease, FSH, LSH, tetanus toxoid, diptheria toxoid, glucagon-like peptide 1, TGF-β, α-IFN, β-IFN, γ-IFN, TNF, lymphotoxin, Migration inhibition factor, neuregulin, CD2, CD3, CD4, CD5, CD6, CD7, CD8, CD11a, CD11b, CD11c, CD13, CD14, CD18, CD19, CE20, CD22, CD23, CD27, CD28, B7.1, B7.2, B7.3, CD29, CD30, CD40, gp39, CD44, CD45, Cdw52, CD56, CD58, CD69, CD72, CTLA-4, LFA-1, SLex, SLey, SLea, SLeb, VLA-1, VLA-2, VLA-3, VLA-4, VLA-5, VLA-6, LFA-1, Mac-1, p150, p95, L-selectin, P-selectin, E-selectin, VCAM-1, ICAM-1, ICAM-2, LFA-3, IL-1, IL-2, IL-3, IL-4, IL-5, IL-6, IL-7, IL-8, IL-9, IL-10, IL-11, IL-12, IL-13, IL-14, IL-15, IL-1R, IL-2R, IL-4R, IL-5R, IL-6R, IL-7R, IL-8R, IL-10R, IL-11R, IL-12R, IL-13R, IL-14R, IL-15R, PF4, MIP1a, MCP1, NAP-2, Groα, Groβ, IL-8, TNF α, TGF β, TSH, VEGF/VPF, PTHrP, EGF, PDGF, endothelin, gastrin releasing peptide (GRP), TNFαR, RGFβ R, TSHR, VEGFR/VPFR, FGFR, EGFR, PTHrPR, PDGFR, EPO-R, GCSF-R, IFNα R, IFNβR, IFNγR, IgE, FceRI, FceRII, complement C3b, complement C5a, complement C5b-9, Rh factor, fibrinogen, fibrin, myelin associated growth inhibitor, prolactin, placental lactogen, thrombopoietin, oncostatin M, ciliary neurotrophic factor (CNTF), leukemia inhibitory factor (LIF), epsilon interferon, omega interferon, tau interferon, granulocyte-colony stimulating factor (G-CSF), granulocyte-macrophage colony stimulating factor (GM-CSF), macrophage colony stimulating factor (M-CSF), and cardiotrophin-1 (CT-1).

14. The method of claim 11, wherein the protein is selected from the group consisting of recombinant human growth hormone (rhGH) and xylanase.

15. The method of claim 11, wherein the protein is rhGH and the crystals have a hexagonal morphology.

16. The method of claim 1, wherein the solution further comprises a buffer and a salt.

17. The method of claim 16, wherein the salt is selected from the group consisting of magnesium chloride, sodium chloride and sodium acetate.

18. The method of claim 1, further comprising a step selected from the group consisting of: changing the pH of the solution, changing the temperature of the solution, changing the dielectric constant of the solution, changing the viscosity of the solution, changing the ionic strength of the solution, changing the concentration of the molecule, adding a reducing agent to the solution, adding an oxidizing agent to the solution, adding a nucleant to the solution, adding a metal ion to the solution, adding a detergent to the solution and adding an amphiphile to the solution.

19. The method of claim 1, wherein the precipitating agent is selected from the group consisting of sodium chloride, sodium acetate, phosphate, citrate, ammonium sulfate, ethanol, glycine-HCl, hydroxyl ethyl starch, heptane 1,2,3-triol, Polyethylene glycol (PEG), and dextran.

20. The method of claim 1, wherein the precipitating agent is PEG.

21. The method of claim 1, wherein the crystals of the molecule have biological activity.

22. The method of claim 1, wherein the rate of depressurization is from about 100 to about 300 bars/minute.

23. A method of forming crystals of a molecule in a solution, comprising:
(a) adding a precipitating agent to the solution,
(b) applying to the solution a hydrostatic pressure of about 0.1 to about 25 kilobars, and
(c) depressurizing the solution.

24. The method of claim 23, comprising applying to the protein a pressure of about 0.5 to about 10 kilobars.

25. The method of claim 23 comprising applying to the protein a pressure of about 0.75 to about 5 kilobar.

26. The method of claim 23, wherein the crystals are not formed when the solution is at atmospheric pressure.

27. The method of claim 23, wherein the pressure is sufficient to form crystals of the molecule.

28. The method of claim 23, wherein an amorphous precipitate is formed when the solution is at atmospheric pressure.

29. The method of claim 23, wherein the morphology of the crystals formed when the solution is subjected to hydrostatic pressure is different from the morphology of the crystals formed when the solution is at atmospheric pressure.

30. The method of claim 23, wherein the number of crystals formed when the solution is subjected to hydrostatic pressure is greater than the number of crystals formed when the solution is at atmospheric pressure.

31. The method of claim 23, wherein the crystals formed when the solution is subjected to hydrostatic pressure are bigger in size than the crystals formed when the solution is at atmospheric pressure.

32. The method of claim 23, wherein the population of crystals formed when the solution is subjected to hydrostatic pressure is more homogeneous than the population of crystals formed when the solution is at atmospheric pressure.

33. The method of claim 23, wherein the amount of any amorphous precipitate formed is less than about 25% of the amount of the crystals.

34. The method of claim 23, further comprising the step of recovering the crystals.

35. The method of claim 23, wherein the molecule is selected from the group consisting of proteins, DNA, RNA, carbohydrates, peptides and polymers.

36. The method of claim 35, wherein the protein is selected from the group consisting of antibodies, antibody fragments, trophic factors, cytokines, lymphokines, toxoids, growth factors, hormones, human growth hormones, growth hormone family members, nerve growth hormones, fertility hormones, postridical hormones, fusion proteins, glycoproteins, synthetic antigens, recombinant antigens, histocompatibility antigens, viral surface proteins, bone morphogenic proteins, enzymes, blood clotting factors, adhesion molecules, multidrug resistance proteins, interleukins, interleukin receptors, chemokines, interferon receptors, T-cell receptors, blood factors, leukocyte markers, monocyte-macrophage colony stimulating factors, granulocyte colony stimulating factors, integrins, selectins, and lectins.

37. The method of claim 35, wherein the protein is selected from the group consisting of erythropoietin, Factor VIII, insulin, amylin, TPA, dornase-α, α-1-antitrypsin, urease, FSH, LSH, tetanus toxoid, diptheria toxoid, glucagon-like peptide 1, TGF-β, α-IFN, β-IFN, γ-IFN, TNF, lymphotoxin, Migration inhibition factor, neuregulin, CD2, CD3, CD4, CD5, CD6, CD7, CD8, CD11a, CD11b, CD11c, CD13, CD14, CD18, CD19, CE20, CD22, CD23, CD27, CD28, B7.1, B7.2, B7.3, CD29, CD30, CD40, gp39, CD44, CD45, Cdw52, CD56, CD58, CD69, CD72, CTLA-4, LFA-1, SLex, SLey, SLea, SLeb, VLA-1, VLA-2, VLA-3, VLA-4, VLA-5, VLA-6, LFA-1, Mac-1, p150, p95, L-selectin, P-selectin, E-selectin, VCAM-1, ICAM-1, ICAM-2, LFA-3, IL-1, IL-2, IL-3, IL-4, IL-5, IL-6, IL-7, IL-8, IL-9, IL-10, IL-11, IL-12, IL-13, IL-14, IL-15, IL-1R, IL-2R, IL-4R, IL-5R, IL-6R, IL-7R, IL-8R, IL-10R, IL-11R, IL-12R, IL-13R, IL-14R, IL-15R, PF4, MIP1a, MCP1, NAP-2, Groα, Groβ, IL-8, TNF α, TGF β, TSH, VEGF/VPF, PTHrP, EGF, PDGF, endothelin, gastrin releasing peptide (GRP), TNFαR, RGFβR, TSHR, VEGFR/VPFR, FGFR, EGFR, PTHrPR, PDGFR, EPO-R, GCSF-R, IFNα R, IFNβR, IFNγR, IgE, FceRI, FceRII, complement C3b, complement C5a, complement C5b-9, Rh factor, fibrinogen, fibrin, myelin associated growth inhibitor, prolactin, placental lactogen, thrombopoietin, oncostatin M, ciliary neurotrophic factor (CNTF), leukemia inhibitory factor (LIF), epsilon interferon, omega interferon, tau interferon, granulocyte-colony stimulating factor (G-CSF), granulocyte-macrophage colony stimulating factor (GM-CSF), macrophage colony stimulating factor (M-CSF), ardiotrophin-1 (CT-1).

38. The method of claim 35, wherein the protein is selected from the group consisting of recombinant human growth hormone (rhGH) and xylanase.

39. The method of claim 35, wherein the protein is rhGH and the crystals have a hexagonal morphology.

40. The method of claim 23, wherein the solution further comprises a buffer and a salt.

41. The method of claim 40, wherein the salt is selected from the group consisting of magnesium chloride, sodium chloride and sodium acetate.

42. The method of claim 23, further comprising a step selected from the group consisting of: changing the pH of the solution, changing the temperature of the solution, changing the dielectric constant of the solution, changing the viscosity of the solution, changing the ionic strength of the solution, changing the concentration of the molecule, adding a reducing agent to the solution, adding an oxidizing agent to the solution, adding a nucleant to the solution, adding a metal ion to the solution, adding a detergent to the solution and adding an amphiphile to the solution.

43. The method of claim 23, wherein the precipitating agent is selected from the group consisting of sodium chloride, sodium acetate, phosphate, citrate, ammonium sulfate, ethanol, glycine-HCl, hydroxyl ethyl starch, heptane 1,2,3-triol, PEG, and dextran.

44. The method of claim 23, wherein the precipitating agent is PEG.

45. The method of claim 23, wherein the crystals of the molecule have biological activity.

46. A method for purifying a composition comprising crystals of a molecule and amorphous precipitate of the molecule comprising:
(a) applying hydrostatic pressure to the composition comprising the crystals and the amorphous precipitate to dissolve at least a portion of the amorphous precipitate while maintaining at least a portion of the crystals, and
(b) depressurizing the composition.

47. The method of claim 46, wherein the amount of crystals is greater after the hydrostatic pressure is applied than before the hydrostatic pressure is applied.

48. The method of claim 46 further comprising a step selected from the group consisting of: adding a precipitating agent to the solution, changing the pH of the solution, changing the temperature of the solution, changing the dielectric constant of the solution, changing the viscosity of the solution, changing the ionic strength of the solution, changing the concentration of the molecule, adding a reducing agent to the solution, adding an oxidizing agent to the solution, adding a nucleant to the solution, adding a metal ion to the solution, adding a detergent to the solution, and adding an amphiphile to the solution.

49. The method of claim 46, wherein the rate of depressurization is from about 100 bars per minute to about 300 bars per minute.

50. The method of claim 46, wherein the crystals are biologically active.

51. The method of claim 46, wherein more than about 20% of the amorphous precipitate is dissolved.

52. A method for purifying a composition comprising crystals of a molecule and amorphous precipitate of the molecule comprising:
(a) applying to the composition comprising the crystals and the amorphous precipitate a pressure from about 0.1 to about 25 kilobars.
(b) depressurizing the composition.

53. The method of claim 52, wherein the pressure is from about 0.5 to about 10 kilobars.

54. The method of claim 52, wherein the pressure is from about 0.75 to about 5 kilobars.

55. The method of claim 52, wherein the rate of depressurization is from about 100 to about 300 bars per minute.

56. The method of claim 52, wherein the crystals are biologically active.

57. The method of claim 52, wherein more than about 20% of the amorphous precipitate is dissolved.

58. A composition comprising crystals made by the method of claim 1.

59. A composition comprising crystals made by the method of claim 23.

60. A pharmaceutical composition comprising crystals made by the method of claim 1 and a pharmaceutically acceptable excipient.

61. A pharmaceutical composition comprising crystals made by the method of claim 23 and a pharmaceutically acceptable excipient.

62. A method of treatment of a subject comprising the step of administering to the subject a therapeutic formulation comprising the crystals made by the method of claim 1.

63. A method of treatment of a subject comprising the step of administering to the subject a therapeutic formulation comprising the crystals made by the method of claim 23.
